(19) United States
(12) Patent Application Publication
(10) Pub. No.: US 2017/0348449 A1
(43) Pub. Date: Dec. 7, 2017

(54) METHOD OF DISINFECTING A THERMAL CONTROL UNIT

(71) Applicant: Stryker Corporation, Kalamazoo, MI (US)

(72) Inventors: Matthew Alan WARD, Farmington Hills, MI (US); Brian Lee ORWAT, Galesburg, MI (US); David SCHARICH, III, Saint Joseph, MI (US)

(21) Appl. No.: 15/611,048

(22) Filed: Jun. 1, 2017

Related U.S. Application Data
(60) Provisional application No. 62/344,779, filed on Jun. 2, 2016.

(51) Int. Cl. A61L 2/18; A61L 2/26

(52) U.S. Cl. CPC A61L 2/18; A61L 2/26; A61L 2202/24; A61L 2202/14

(57) ABSTRACT

A method of disinfecting a fluid circuit of a thermal control for delivering temperature controlled fluid to at least one patient therapy device comprises the steps of providing an aqueous mixture comprising a disinfectant and circulating the aqueous mixture in the thermal control unit to disinfect the fluid circuit. The disinfectant comprises free-chlorine, a phenol, hydrogen peroxide (H₂O₂), or combinations thereof. If the disinfectant comprises free-chlorine, the free-chlorine is provided by a chlorinated isocyanurate (e.g. sodium dichloroisocyanurate; NaDCC). In addition, the free-chlorine is present in the aqueous mixture in an amount of at least about 100 parts per million (ppm). If the disinfectant comprises the phenol, the phenol is natural (e.g. thymol). In addition, the phenol is present in the aqueous mixture in an amount of at least about 10,000 ppm. If utilized, H₂O₂ is present in the aqueous mixture in an amount of at least about 5,000 ppm.

### CROSS-REFERENCE TO RELATED APPLICATIONS

This application claims priority to and all advantages of U.S. Provisional Patent Application No. 62/344,779 filed on 2 Jun. 2016, the content of which is incorporated herein by reference.

### FIELD OF THE INVENTION

The present invention generally relates to a method and to a system, and more specifically to a method of disinfecting a fluid circuit of a thermal control unit and to a system comprising the thermal control unit. The thermal control unit is for delivering temperature controlled fluid to at least one patient therapy device.

### BACKGROUND OF THE INVENTION

Thermal control systems are commercially available from a number of companies and utilized for controlling the temperature of a patient by supplying temperature-controlled fluid (e.g. water) to one or more patient therapy devices (e.g. pads, blankets, wraps, or similar structures) that are positioned in contact with, or adjacent to, a patient. The temperature of the fluid is controlled by a thermal control unit that provides fluid to the patient therapy device(s). After passing through the patient therapy device(s), the fluid is returned to the thermal control unit where any necessary adjustments to the returning fluid temperature are made before being pumped back to the patient therapy device(s). In some instances, the temperature of the fluid is controlled to a target temperature, while in other instances the temperature of the fluid is controlled in order to effectuate a change or steady-state patient temperature.

Health care regulatory agencies have identified a need for improved disinfection of fluid circuits, thermal control units and systems, and other related components in an effort to prevent patients from becoming ill via spread of pathogens (e.g. bacteria, microorganisms, etc.). Bleach (e.g. sodium hypochlorite; NaOCl) is commonly used to generate free-chlorine for disinfection but presents material compatibility issues. Specifically, sodium hydroxide (NaOH) is generated via NaOCl hydrolysis, which corrodes/attack the fluid circuit and other components of the thermal control system over time. In addition, bleach can irritate the skin or lungs, which is especially problematic when handling or working with certain patients. Other disinfectants fail to provide adequate levels of disinfection, are difficult to handle, and/or are cost prohibitive.

In view of the foregoing, there remains an opportunity to provide improved methods of disinfecting fluid circuits, thermal control units, and systems. There also remains an opportunity to provide improved fluid circuits, thermal control units, and systems.

### BRIEF SUMMARY OF THE INVENTION

A method of disinfecting a fluid circuit of a thermal control unit is provided. The thermal control unit is for delivering temperature controlled fluid to at least one patient therapy device. The method comprises the step of providing an aqueous mixture. The aqueous mixture comprises a disinfectant. The method further comprises the step of circulating the aqueous mixture in the thermal control unit to disinfect the fluid circuit. The disinfectant comprises free-chlorine, a phenol, hydrogen peroxide (H₂O₂), or combinations thereof. If the disinfectant comprises free-chlorine, the free-chlorine is provided by a chlorinated isocyanurate. In addition, the free-chlorine is present in the aqueous mixture in an amount of at least about 100 parts per million (ppm). If the disinfectant comprises the phenol, the phenol is natural. In addition, the phenol is present in the aqueous mixture in an amount of at least about 10,000 ppm. If the disinfectant comprises H₂O₂, the H₂O₂ is present in the aqueous mixture in an amount of at least about 5,000 ppm.

A system is also provided. The system comprises a thermal control unit. The thermal control unit has a fluid circuit for delivering temperature controlled fluid to at least one patient therapy device. An aqueous mixture is disposed in the fluid circuit. The aqueous mixture comprises a disinfectant for disinfecting the fluid circuit. The aqueous mixture and disinfectant are as described above for the method.

Before the various embodiments disclosed herein are explained in detail, it is to be understood that the claims are not to be limited to the details of operation or to the details of construction, nor to the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of steps or components. Nor should the use of enumeration be construed as excluding from the scope of the claims any additional steps or components that might be combined with or into the enumerated steps or components.

### BRIEF DESCRIPTION OF THE DRAWINGS

FIG. 1A is a schematic of a thermal control unit having a circulation channel, an inlet and an outlet, with a patient therapy device connected between the outlet and inlet;

FIG. 1B is a schematic of the fluid circuit of the thermal control unit of FIG. 1A with the patient therapy device removed;

FIG. 2 is a schematic of another thermal control unit having a circulation channel, an inlet and an outlet, with a bypass line connected between the outlet and inlet;

FIG. 3 is a schematic of a fluid circuit of a thermal control unit having a circulation channel, an inlet, an outlet, and a reservoir, with a bypass line routed from the outlet to the reservoir;

FIG. 4 is a schematic of another thermal control unit having a circulation channel, an inlet, an outlet, and a reservoir, with a first bypass line routed from the outlet to the reservoir and a second bypass line connected between the outlet and inlet;

FIG. 5 is a schematic of another thermal control unit having a circulation channel, an inlet, an outlet, and a reservoir, with a bypass line routed from the outlet to a dispenser for providing a disinfectant, and the dispenser routed to the reservoir;

FIG. 6 is a schematic of another thermal control unit having a circulation channel, an inlet, an outlet, and a reservoir, with a first bypass line routed from the outlet to a dispenser for providing a disinfectant, the dispenser routed to the reservoir, and a second bypass line connected between the outlet and inlet;

FIG. 7 is a schematic of another thermal control unit having a circulation channel, an inlet, an outlet, and a reservoir, with a bypass line connected between the outlet and inlet, and a dispenser for providing a disinfectant routed to the reservoir;

FIG. 8 is a schematic of another thermal control unit having a circulation channel, an inlet, an outlet, and a reservoir, with a bypass line routed from the outlet to the reservoir, and a dispenser for providing a disinfectant at least partially disposed in the reservoir;

FIG. 9 is a schematic of another thermal control unit having a circulation channel, an inlet, an outlet, and a reservoir, with a bypass line connected between the outlet and inlet, and a dispenser for providing a disinfectant connected to the circulation channel;

FIG. 10 is a schematic of another thermal control unit having a circulation channel, an inlet, an outlet, and a reservoir, with a bypass line connected between the outlet and inlet, and an ultraviolet (UV) light disposed adjacent a separator for providing UV disinfection;

FIG. 11 is a schematic of another thermal control unit having a circulation channel, an inlet, an outlet, and a reservoir, with a bypass line connected between the outlet and inlet, and an ozone (O₃) generator connected to the circulation channel for providing ozone disinfection;

FIG. 12 is a schematic of another thermal control unit that may be disinfected by the method disclosed herein;

FIG. 13A is a schematic of another thermal control unit that may be disinfected by the method disclosed herein, with the thermal control unit flowing in a heating mode;

FIG. 13B is a schematic of the thermal control unit of FIG. 13A, with the thermal control unit flowing in a cooling mode;

FIG. 14 is a schematic of another thermal control unit that may be disinfected by the method disclosed herein; and

FIG. 15 is a rear view of another thermal control unit that may be disinfected by the method disclosed herein.

### DETAILED DESCRIPTION OF THE EMBODIMENTS

A method of disinfecting a fluid circuit of a thermal control unit is provided (referred to hereinafter as the “method”). The thermal control unit is for delivering temperature controlled fluid to at least one patient therapy device. The patient therapy device may also be referred to as a thermal transfer device (TTD) or a heater-cooler system/device, such as those used to treat hypothermia, hyperthermia, and/or during open heart surgeries. The thermal control unit is described in greater detail further below. The thermal control unit may be (or may be part of) a thermal management system or a temperature management system. Such systems are understood in the healthcare art.

The method comprises the step of providing an aqueous mixture. The aqueous mixture comprises a disinfectant. The method further comprises the step of circulating the aqueous mixture in the thermal control unit to disinfect the fluid circuit. The disinfectant comprises free-chlorine, a phenol, hydrogen peroxide (H₂O₂), or combinations thereof. If a combination of disinfectants is utilized, the combination may comprise two or all three of the disinfectants, e.g. free-chlorine and the phenol, free-chlorine and H₂O₂, the phenol and H₂O₂, or free-chlorine, the phenol, and H₂O₂. Otherwise, one of the disinfectants can be used alone, e.g. free-chlorine to the exclusion of the phenol and H₂O₂.

The minimum amount of disinfectant utilized for disinfecting the fluid circuit can be readily determined via routine experimentation depending on, for example, the particular thermal control unit, the desired outcome, etc. In general, the disinfectant should be present in the aqueous mixture in a sufficient amount to initially disinfect or “shock” the fluid circuit. The exact amount of the disinfectant can be “dialed in” to an appropriate level for a given situation. Utilizing an insufficient amount of the disinfectant may not achieve the desired level of disinfection, whereas utilizing an excess amount of the disinfectant may add unnecessary cost.

Various levels of disinfectant activity may be desired including low-level, intermediate-level, or high-level disinfection. These levels of disinfection are generally understood in the healthcare art and are often defined, established, controlled, or mandated by the Centers for Disease Control and Prevention (CDC), the Food and Drug Administration (FDA), or the Environmental Protection Agency (EPA). For example, specific definitions for certain disinfection levels are described in Technical Information Report (TIR) 12:2010 published by the Association for the Advancement of Medical Instrumentation (AAMI). The disclosure of AAMI TIR12:210 is incorporated herein by reference in its entirety and includes the following definitions:
a) Low-level disinfection kills vegetative forms of bacteria, some fungi, and lipid viruses. Low-level disinfection cannot be relied on to destroy mycobacteria, bacterial endospores, or small nonlipid viruses.
b) Intermediate-level disinfection kills viruses, mycobacteria, fungi, and vegetative bacteria, but not necessarily bacterial spores.
c) High-level disinfection kills all microbial organisms, but not necessarily large numbers of bacterial spores.

In a first general embodiment (referred to hereinafter as the “first embodiment”), the disinfectant comprises free-chlorine. In further embodiments, the disinfectant consists essentially of free-chlorine, or alternatively consists of (or is) free-chlorine. In various embodiments, the free-chlorine comprises hypochlorous acid (HOCl), hypochlorite ions (OCL⁻), or a mixture thereof. In certain embodiments, the free-chlorine comprises a mixture of HOCl and OCL⁻, a majority of HOCl, or a majority of OCL⁻. Free-chlorine may also be referred to as free available chlorine (FAC).

As used herein, the phrase “consisting essentially of” generally encompasses the specifically recited elements/components for a particular embodiment. Further, the phrase “consisting essentially of” generally encompasses and allows for the presence of additional (or optional) components that do not materially impact the basic and/or novel characteristics of that particular embodiment. In certain embodiments, the phase “consisting essentially of” allows for the presence of ≦10, ≦5, or ≦1, weight percent (wt %) of additional, secondary, or optional components based on the total weight of the primary component(s).

In the first embodiment, the free-chlorine is present in the aqueous mixture in an amount of at least about 100 parts per million (ppm). As understood in the art, 10,000 ppm is equal to 1%. As such, the ppm amounts herein can readily be converted to % amounts or vice versa. In various embodiments, the free-chlorine is present in the aqueous mixture in an amount of from about 100 ppm to about 10,000 ppm, alternatively in an amount of from about 250 ppm to about 5,000 ppm, alternatively in an amount of from about 500 ppm to about 2,500 ppm, alternatively in an amount of from about 750 ppm to about 2,250, alternatively in an amount of from about 1,000 ppm to about 2,000 ppm, or alternatively in an amount of about 2,000 ppm. Various ranges and subranges of these amounts are also contemplated. In addition, these amounts can be adjusted to account for inclusion of the phenol and/or H₂O₂ in embodiments including a combination of disinfectants.

In certain embodiments, the free-chlorine is present in the aqueous mixture in an amount of at least about 2,000 ppm. Without being bound or limited to any particular theory, it is thought that 2,000 ppm of free-chlorine (or thereabout) is especially useful for achieving intermediate-level disinfection of the fluid circuit. It is to be appreciated that the amount of free-chlorine in the aqueous mixture will gradually decrease with the passage of time. As such, the ppm amounts described above may be referred to as initial amounts, formed amounts, loaded amounts, or use/application amounts.

The free-chlorine is provided by a chlorinated isocyanurate. In general, a majority of, or an entirety of, the free-chlorine is provided by the chlorinated isocyanurate. In various embodiments, the chlorinated isocyanurate is selected from the group consisting of mono, di and trichloro isocyanurates. Examples of suitable chlorinated isocyanurates include sodium dichloroisocyanurate (NaDCC, anhydrous), sodium dichloroisocyanurate dihydrate (NaDCC·2H₂O), potassium dichloroisocyanurate (KDCC), trichloroisocyanuric acid (TCCA), and combinations thereof. Suitable chlorinated isocyanurates are commercially available from a number of suppliers, including from ACTIVON®, BRULIN® (Brulin & Co.), Hydrachem Ltd., MEDENTECH® (Medentech Ltd.), Occidental Chemical Corporation (OxyChem), SIGMA-ALDRICH®, etc.

In certain embodiments, the chlorinated isocyanurate comprises NaDCC. NaDCC is generally of the chemical formula C₃Cl₂N₃NaO₃, is of CAS Number 2893-78-9, and may also be referred to as sodium 3,5-dichloro-2,4,6-trioxo-1,3,5-triazinan-1-ide, sodium troclosene, sodic troclosene, troclosenum natricum, dichloroisocyanuric acid, sodium salt, or sodium salt of dichloroisocyanuric acid. In general, approximately 1.6 mg of NaDCC delivers about 1 mg FAC per liter of water. One of skill in the art can readily calculate dosing of NaDCC depending on, for example, the NaDCC source (e.g. purity or wt %) and desired ppm of free-chlorine.

Without being bound or limited to any particular theory, it is thought that NaDCC provides a number of advantages over other free-chlorine generators or other disinfectants. For example, NaDCC has increased compatibility with materials of construction of the fluid circuit or the thermal control unit. For example, bleach or residue(s) thereof (e.g. NaOH) can cause damage to materials of construction. NaDCC also has increased ease of shipping, storage, and handling. Relative to liquid bleach, powdered or dry NaDCC is generally safer to handle. In addition, NaDCC provides a free-chlorine “sink,” which buffers the aqueous mixture for a period of time (e.g. for about a day to about one week). Moreover, NaDCC is approved by the EPA.

Free-chlorine is an active antimicrobial compound. In general, it is thought that three things can happen when free-chlorine is added to (or formed in) water:
1) Some free-chlorine reacts through oxidization with organic matter and pathogens in the water and kills them. This portion is called “consumed chlorine.”
2) Some free-chlorine reacts with other organic matter and forms new chlorine compounds. This portion is called “combined chlorine.”
3) Excess free-chlorine that is not consumed or combined remains in the water. This portion is called “free residual chlorine” (FRC). The FRC helps prevent recontamination of the treated/disinfected water.

In a second general embodiment (referred to hereinafter as the “second embodiment”), the disinfectant comprises the phenol. In further embodiments, the disinfectant consists essentially of the phenol, or alternatively consists of (or is) the phenol.

In the second embodiment, the phenol is present in the aqueous mixture in an amount of at least about 10,000 ppm. In various embodiments, the phenol is present in the aqueous mixture in an amount of from about 10,000 ppm to about 500,000 ppm, alternatively in an amount of from about 25,000 ppm to about 400,000 ppm, alternatively in an amount of from about 50,000 ppm to about 300,000 ppm, alternatively in an amount of from about 75,000 ppm to about 200,000 ppm, alternatively in an amount of from about 100,000 ppm to about 150,000 ppm, or alternatively in an amount of about 130,000 ppm. Various ranges and subranges of these amounts are also contemplated. In addition, these amounts can be adjusted to account for inclusion of the free-chlorine and/or H₂O₂ in embodiments including a combination of disinfectants. For example, if about equal amounts of free-chlorine and phenol are desired (i.e., 1:1), a weighted average calculation can be utilized to determine respective amounts such as (0.5*100 ppm free-chlorine)+(0.5*10,000 ppm phenol). Other ratios and mixtures of two or more of the disinfectants can also be readily calculated and such mixtures and ratios are contemplated.

In certain embodiments, the phenol is present in the aqueous mixture in an amount of at least about 130,000 ppm. Without being bound or limited to any particular theory, it is thought that 130,000 ppm of the phenol (or thereabout) is especially useful for achieving intermediate-level disinfection of the fluid circuit. It is to be appreciated that the amount of phenol in the aqueous mixture will gradually decrease with the passage of time. As such, the ppm amounts described above may be referred to as initial amounts, formed amounts, loaded amounts, or use/application amounts.

The phenol utilized for the method of this disclosure is natural. That is to say that the phenol utilized herein is naturally sourced (or naturally occurring) rather than being synthetic or man-made. In general, the phenol is extractable from plant material. The method is not limited to a particular extraction method of the phenol and suitable natural phenols for the method are commercially available from a number of suppliers, including from SIGMA-ALDRICH®, Wexford Labs, Inc., etc. If desired, there are a variety of extraction methods that may be used to produce phenols suitable for the method. These extraction methods include, but are not limited to, the extraction methods disclosed in U.S. Pat. No. 7,897,184, which is incorporated herein by reference for this purpose.

In certain embodiments, the phenol comprises thymol. In general, the phenol consists essentially of, or consists of, thymol. Thymol is generally of the chemical formula C₁₀H₁₄O, is of CAS Number 89-83-8, and may also be referred to as 2-isopropyl-5-methylphenol (IPMP) or 5-methyl-2-isopropyl-1-phenol. Thymol may be extracted from common thyme (Thymus vulgaris) and/or other plants including Euphrasia rostkoviana, Monarda didyma, Monarda fistulosa, Trachyspermum ammi, Origanum compactum, Origanum dictamnus, Origanum onites, Origanum vulgare, Thymus glandulosus, Thymus hyemalis, Thymus zygis, and combinations thereof. Again, however, it is to be appreciated that suitable natural phenols for the method (including thymol) are commercially available. As such, this disclosure is not limited to a particular extraction method or source of the phenol/thymol.

Without being bound or limited to any particular theory, it is thought that thymol provides a number of advantages over other disinfectants. For example, thymol has increased compatibility with materials of construction of the fluid circuit or the thermal control unit. Thymol also has increased ease of shipping, storage, and handling. In addition, natural thymol provides a “green” option. Moreover, thymol is approved by the EPA.

In a third general embodiment (referred to hereinafter as the “third embodiment”), the disinfectant comprises H₂O₂. In further embodiments, the disinfectant consists essentially of H₂O₂, or alternatively consists of (or is) H₂O₂.

In the third embodiment, the H₂O₂ is present in the aqueous mixture in an amount of at least about 5,000 ppm (i.e., 0.5%). In various embodiments, the H₂O₂ is present in the aqueous mixture in an amount of from about 5,000 ppm to about 100,000 ppm, alternatively in an amount of from about 5,000 ppm to about 80,000 ppm, alternatively in an amount of from about 5,000 ppm to about 50,000 ppm, alternatively in an amount of from about 5,000 ppm to about 40,000, alternatively in an amount of from about 5,000 ppm to about 30,000 ppm, alternatively in an amount of from about 5,000 ppm to about 20,000 ppm, alternatively in an amount of from about 5,000 ppm to about 10,000 ppm, alternatively in an amount of about 5,000 ppm. Various ranges and subranges of these amounts are also contemplated. In addition, these amounts can be adjusted to account for inclusion of the free-chlorine and/or phenol in embodiments including a combination of disinfectants. For example, if about equal amounts of free-chlorine, phenol, and H₂O₂ are desired (i.e., 1:1:1), a weighted average calculation can be utilized to determine respective amounts such as (0.33*100 ppm free-chlorine)+(0.33*10,000 ppm phenol)+(0.33*5,000 ppm H₂O₂). Other ratios and mixtures of two or more of the disinfectants can also be readily calculated and such mixtures and ratios are contemplated.

In certain embodiments, the H₂O₂ is present in the aqueous mixture in an amount of at least about 5,000 ppm. Without being bound or limited to any particular theory, it is thought that 5,000 ppm of H₂O₂ (or thereabout) is especially useful for achieving low-level disinfection of the fluid circuit and/or for preventing anti-microbial activity in the fluid circuit. It is to be appreciated that the amount of H₂O₂ in the aqueous mixture will gradually decrease with the passage of time. As such, the ppm amounts described above may be referred to as initial amounts, formed amounts, loaded amounts, or use/application amounts.

The H₂O₂ can be provided by various compounds. In various embodiments, the H₂O₂ is provided by a perhydrate. In general, a majority of, or an entirety of, the H₂O₂ is provided by the perhydrate. Suitable perhydrates include adducts of percarbonate salts, such as sodium percarbonate. Suitable perhydrates are commercially available from a number of suppliers, including from SIGMA-ALDRICH®, Solvay Chemicals, Inc., etc.

In certain embodiments, the H₂O₂ is provided by sodium percarbonate. Sodium percarbonate is generally of the chemical formula Na₂CO₃·1.5H₂O₂ (or 2Na₂CO₃·3H₂O₂), is of CAS Number 15630-89-4, and may also be referred to as sodium carbonate-hydrogen peroxide (2/3), sodium carbonate sesquiperhydrate, PCS (percarbonate de soude), solid hydrogen peroxide, sodium carbonate hydrogen peroxide, or sodium carbonate peroxyhydrate (SCP). In general, approximately 3 mg of sodium percarbonate delivers about 1 mg H₂O₂ per liter of water. One of skill in the art can readily calculate dosing of H₂O₂ depending on, for example, the H₂O₂ source (e.g. purity or wt %) and desired ppm of H₂O₂.

Without being bound or limited to any particular theory, it is thought that H₂O₂ provides a number of advantages over other disinfectants. For example, H₂O₂ has increased compatibility with materials of construction of the fluid circuit or the thermal control unit. That being said, if too much H₂O₂ is present (e.g. >30,000 ppm), certain material compatibility issues way arise, e.g. with copper. As for sodium percarbonate, it has increased ease of shipping, storage, and handling. That being said, if too much sodium percarbonate is utilized, excessive generation of H₂O₂ (e.g. ≧80,000 ppm) may warrant increased safety protocols during handling of the aqueous mixture to prevent the possibility of chemical burns or irritation. H₂O₂ also provides a “green” option because it readily degrades. Moreover, H₂O₂ is approved by the EPA.

The aqueous mixture comprises water in addition to the disinfectant. In various embodiments, the aqueous mixture consists essentially of the disinfectant and water, or alternatively consists of (or is) the disinfectant and water. Different types of water can be utilized including tap water and purified water. In various embodiments, the water is purified water so as to not contaminate the fluid circuit or thermal control unit. Purification processes are understood in the art and can include distillation, deionization, demineralization, and other processes, e.g. reverse osmosis, carbon filtration, microporous filtration, ultrafiltration, ultraviolet (UV) oxidation, electro-dialysis, etc. This disclosure is not limited to a particular purification process or source of purified water. In certain embodiments, the water of the aqueous mixture comprises distilled water.

The aqueous mixture can be of various volumes, but the volume generally complements the volume of the fluid circuit and/or the volumetric capacity of the thermal control unit. The aqueous mixture can also be of a lower or higher volume than that of the fluid circuit volume. In various embodiments, the aqueous mixture has a volume of at least about 1 liter. In certain embodiments, the aqueous mixture has a volume of from about 1 liter to about 30 liters, alternatively a volume of from about 1.5 liters to about 20 liters, alternatively a volume of from about 2 liters to about 10 liters, alternatively a volume of from about 3 liters to about 5 liters, or alternatively a volume of from about 3.75 liters to about 4.25 liters. In specific embodiments, the aqueous mixture has a volume of about 1 gallon. In other embodiments, the aforementioned volumes are increased by about 50%, alternatively are increased by about 100%, alternatively are increased by about 150% (or more) to account for attachments (e.g. patient therapy devices and/or lines/hoses) or other components of the thermal control unit (e.g. tanks and/or reservoirs). Various ranges and subranges of these volumes are also contemplated.

The aqueous mixture can be of various pH, but the pH is generally not overly caustic or overly alkaline to prevent (or lessen) material of constriction compatibility or handling concerns. In addition, overly caustic or overly alkaline pH may reduce the effectiveness of the disinfectant and/or promote formation of undesirable byproducts in the aqueous mixture. In various embodiments, the aqueous mixture has a pH of from about 6 to about 9, alternatively a pH of from about 6.5 to about 8.5, alternatively a pH of from about 7 to about 8, or alternatively a pH of from about 7 to about 7.5. Various ranges and subranges of these pH values are also contemplated.

The aqueous mixture can be of various temperatures, but the temperature generally compliments the ambient (or operating) temperature of the thermal control unit or space having the thermal control unit. The aqueous mixture can also be of a temperature lower or higher than that of ambient. Many thermal control units in the art can operate between about 0° C. and about 100° C., alternatively between about 4° C. and about 40° C. Room temperature is generally between about 23° C. and about 25° C. In various embodiments, the aqueous mixture has a temperature of from about 5° C. to about 95° C., alternatively a temperature of from about 10° C. to about 75° C., alternatively a temperature of from about 15° C. to about 55° C., alternatively a temperature of from about 20° C. to about 35° C., alternatively a temperature of from about 23° C. to about 27° C., alternatively a temperature of from about 23° C. to about 25° C., or alternatively a temperature of about 25° C. Various ranges and subranges of these temperatures are also contemplated. In general, active heating or cooling of the aqueous mixture via the thermal control unit is not required. However, it is to be appreciated that components of the thermal control unit, e.g. a pump, may impart minimal thermal energy to the aqueous mixture while being circulated. As such, active cooling may be utilized to maintain (near) consistent temperature during circulation although this is not required.

In certain embodiments, the aqueous mixture further comprises an additive. The additive can be of various types understood in the art. Examples of suitable additives include those selected from the group consisting of surfactants, builders, activators, inhibitors, solubilizers, descalers, chelating agents, acids, bases, water conditioning agents, pH buffers, etc., and combinations thereof. If utilized, the additive(s) can be present in the aqueous mixture in various amounts. In various embodiments, the additive comprises at least one surfactant. Examples of suitable surfactants include nonionic surfactants, anionic surfactants, cationic surfactants, amphoteric surfactants, and combinations thereof. Suitable additives and amounts thereof can be readily determined via routine experimentation depending on, for example, the particular additive, the desired outcome, etc. Utilizing the additive is optional and this disclosure is not limited to a particular additive or amount thereof.

The aqueous mixture can be provided in various ways. For example, the aqueous mixture can be formed, provided “as is” or further formed from a prior mixture. In certain embodiments, the aqueous mixture may be fully or partially formed outside the fluid circuit and then disposed in the fluid circuit. For example, the disinfectant (and/or disinfectant component) and water can be added to a vessel (in any order) to form the aqueous mixture and then the aqueous mixture is added (e.g. via pouring, injection, etc.) to the fluid circuit. In other embodiments, the aqueous mixture may be partially or fully formed inside the fluid circuit, e.g. in situ. For example, water may already be present in the fluid circuit and the disinfectant (and/or disinfectant component) is added to the fluid circuit (e.g. via pouring, dropping, feeding, dispersing, injection, etc.) to form the aqueous mixture in the fluid circuit.

In general, the aqueous mixture is at least partially formed when the disinfectant (and/or disinfectant component) and water are physically contacted and fully formed once the respective amounts of the disinfectant (and/or disinfectant component) and water are physically contacted. While referred to as an aqueous “mixture,” purposeful mixing is not necessarily required. The aqueous mixture or components thereof can be introduced into the fluid circuit by various points of entry, e.g. via an input, a port, a reservoir, a tank, an attachment, a hose, etc.

In various embodiments, the aqueous mixture is provided by mixing a disinfectant component with water. The disinfectant component comprises the disinfectant (and/or disinfectant generator), e.g. the chlorinated isocyanurate, the phenol, the perhydrate, or combinations thereof. Each of the water, chlorinated isocyanurate, phenol, and perhydrate are generally as described above. For example, the chlorinated isocyanurate can be NaDCC, the phenol can be thymol, the perhydrate can be sodium percarbonate, and the water can be distilled water.

The disinfectant component can be of different forms. In various embodiments, the disinfectant component is in the form of a solid, a liquid, or a combination thereof. In certain embodiments, the disinfectant component is in the form of a tablet, a granule, a powder, a liquid, or combinations thereof. Certain forms may be more useful for storage and handling (e.g. solid forms) while other forms may be more useful for forming the aqueous mixture (e.g. powdered or liquid forms).

In various embodiments, generally those where the disinfectant component is a solid, the disinfectant component further comprises at least one effervescent compound such that the disinfectant component effervesces to facilitate formation of the aqueous mixture. Effervescent compounds are understood in the art and are generally recognized by their ability to react and release a gas while in solution. This is especially useful when the disinfectant component is in the form of a solid, e.g. a tablet and/or granule, to facilitate getting the disinfectant into solution. The effervescent effect or esservescence (if still present), may also help to maximize contact areas, reaching nooks and corners of the fluid circuit that are not normally easily reachable or accessible. Alternatively or in addition, including one or more surfactants and/or foaming agents may also have a similar effect or benefit.

Acids and bases are common effervescent compounds and there is generally one of each for reaction and release of reaction products (e.g. carbon dioxide). Examples of suitable acids include citric acid, malic acid, tartaric acid, adipic acid, fumaric acid, and combinations thereof. Examples of suitable bases include sodium bicarbonate, potassium bicarbonate, sodium carbonate, potassium carbonate, and combinations thereof. In various embodiments, the disinfectant component comprises adipic acid, sodium carbonate, and the disinfectant (and/or disinfectant generator). In further embodiments, the disinfectant component consists essentially of an acid, a base, and the disinfectant (and/or disinfectant generator), alternatively the disinfectant component consists essentially of adipic acid, sodium carbonate, and the chlorinated isocyanurate (e.g. NaDCC).

In certain embodiments, the acid for effervescing (e.g. adipic acid) is present in the disinfectant component in an amount of from about 5 wt % to about 50 wt %, alternatively in an amount of from about 10 wt % to about 40 wt %, alternatively in an amount of from about 15 wt % to about 30 wt %, alternatively in an amount of from about 20 wt % to about 25 wt %, or alternatively in an amount of from about 21 wt % to about 23 wt %. Various ranges and subranges of these amounts are also contemplated.

In further embodiments, the base for effervescing (e.g. sodium carbonate) is present in the disinfectant component in an amount of from about 0.5 wt % to about 20 wt %, alternatively in an amount of from about 1 wt % to about 15 wt %, alternatively in an amount of from about 2 wt % to about 10 wt %, alternatively in an amount of from about 3 wt % to about 5 wt %, or alternatively in an amount of from about 4 wt % to about 5 wt %. Various ranges and subranges of these amounts are also contemplated.

In yet further embodiments, the disinfectant (and/or disinfectant generator, e.g. NaDCC) is present in the disinfectant component in an amount of from about 25 wt % to about 75 wt %, alternatively in an amount of from about 30 wt % to about 70 wt %, alternatively in an amount of from about 35 wt % to about 65 wt %, alternatively in an amount of from about 40 wt % to about 60 wt %, or alternatively in an amount of from about 45 wt % to about 55 wt %. Various ranges and subranges of these amounts are also contemplated.

In certain embodiments where the disinfectant comprises H₂O₂, one or more accelerators may also be utilized. For example, an acid, such as peracetic acid and/or phosphoric acid, may be utilized to accelerate the formation and/or efficacy of H₂O₂. Suitable accelerated hydrogen peroxides (AHPs), or components thereof, are disclosed in U.S. Pat. No. 7,354,604; U.S. Pat. No. 7,632,523; and U.S. Pat. No. 9,233,180; and in U.S. Patent App. Pub. No. 2012/0230869 and U.S. Patent App. Pub. No. 2014/0044596; each of which is incorporated herein by reference in its entirety. In general, AHPs are useful for making H₂O₂ more effective during short contact times relative to H₂O₂ that is not accelerated. The use of accelerators and AHPs is optional and are generally not required for the method of this disclosure.

Suitable disinfectant components are commercially available from a number of suppliers, including from ACTIVON®, BRULIN®, Hydrachem Ltd., MEDENTECH®, OxyChem, SIGMA-ALDRICH®, Solvay Chemicals, Inc., Wexford Labs, Inc., etc. Specific examples of suitable disinfectant components include those commercially available from ACTIVON® under the trade names EFFERCEPT™ and EFFERSAN™, such as EFFERSAN™ Multi-Purpose Disinfecting Tablets; from BRULIN® under the trade name BRU-CLEAN™ and BRUTAB™, such as BRU-CLEAN TBC® and BRUTAB6S® Effervescent Disinfectant Tablets; from Hydrachem Ltd. under the trade name BIOSPOT®, such as BIOSPOT® Effervescent Chlorine Tablets; from MEDENTECH® under the trade names AQUASEPT™, AQUATABS™ and KLORSEPT™, such as AQUATABS™ 1000, AQUATABS™ Granules and KLORSEPT™ Bleach Tablets; from OxyChem under the trade name ACL®, such as ACL® 60; and from Wexford Labs, Inc. under the trade name THYMO-CIDE™. Further examples of suitable disinfectants, disinfectant generators, disinfectant components, and optional additives are disclosed in U.S. Pat. No. 7,357,248 and U.S. Patent App. Pub. No. 2012/0015948, each of which is incorporated herein by reference in its entirety.

The aqueous mixture can be circulated in the thermal control unit, and more specifically circulated in the fluid circuit of the thermal control unit, for various amounts of time. The minimum amount of time for disinfecting the fluid circuit can be readily determined via routine experimentation depending on, for example, the particular thermal control unit, the particular aqueous mixture, the desired outcome, etc. In general, the aqueous mixture should be circulated for a sufficient amount of time to initially disinfect or “shock” the fluid circuit. The exact amount of time can be “dialed in” to an appropriate time period for a given situation. Circulating for an insufficient amount of time may not achieve the desired level of disinfection, whereas utilizing an excess amount of time may add unnecessary cost or delay, e.g. by impeding use of the thermal control unit for its intended purpose (e.g. managing a patient's temperature).

Without being bound or limited to any particular theory, it is thought that the aqueous mixture can also sit for a period of time after an initial circulation cycle and still be effective in disinfecting the fluid circuit over the passage of time. However, is thought that circulating the aqueous mixture ensures mixing and improved disinfection especially where the fluid circuit is complex in shape, flow path or design. For example, circulating the aqueous mixture can impart turbulent, transitional, and/or laminar flow in the fluid circuit thereby better ensuring that potential stagnation points or eddies are adequately disinfected. Circulation of the aqueous mixture is also useful for physically cleaning an inner surface of the fluid circuit. For example, scaling or other contaminants (if present) can be physically eroded by the passing/circulating aqueous mixture. Circulation of the aqueous mixture can be ramped up and/or down in flow rate and/or can be pulsed (e.g. by abruptly starting and stopping flow) which may also be useful for ensuring adequate disinfection of the fluid circuit.

In various embodiments, the aqueous mixture is circulated in the fluid circuit at a flow rate of from about 0.5 liters per minute (L/min) to about 15 L/min, alternatively a flow rate of from about 1 L/min to about 10 L/min, or alternatively a flow rate of from about 2 L/min to about 5 L/min. The flow rate can remain constant or vary during circulation of the aqueous mixture. This disclosure is not limited to a particular flow rate of the aqueous mixture.

In various embodiments, the aqueous mixture is circulated in the thermal control unit for a least about 5 minutes. In certain embodiments, the aqueous mixture is circulated in the thermal control unit for about 5 minutes to about 30 minutes, alternatively is circulated in the thermal control unit for about 5 minutes to about 25 minutes, alternatively is circulated in the thermal control unit for about 7.5 minutes to about 20 minutes, alternatively is circulated in the thermal control unit for about 7.5 minutes to about 17.5 minutes, alternatively is circulated in the thermal control unit for about 10 minutes to about 15 minutes, or alternatively is circulated in the thermal control unit for about 10 minutes. Various ranges and subranges of these time periods are also contemplated.

It is to be appreciated that more than one aqueous mixture may be utilized during the shock portion of the method. For example, a first aqueous mixture can be circulated for a period of time (e.g. about 5 minutes), removed, and then a second aqueous mixture can be circulated for an additional period of time (e.g. about 5 minutes). Third or higher aqueous mixtures may also be utilized for periods of time. The aqueous mixtures may be the same or different, typically different. For example, the aqueous mixtures may utilize different disinfectants, different combinations of disinfectants, and/or different amounts of disinfectant(s).

In various embodiments, the method further comprises the step of removing the aqueous mixture from the thermal control unit after circulating the aqueous mixture in the thermal control unit to disinfect the fluid circuit. The aqueous mixture can be removed a short time after circulation, or some time period thereafter. Without being bound or limited to any particular theory, it is thought that the aqueous mixture may be left in the fluid circuit without harming the thermal control unit and can also provide free residual disinfectant (e.g. FRC) for a period of time. However, it is thought that removing the aqueous mixture may be prudent to ensure disinfection of the fluid circuit and/or to ensure protection of the thermal control unit or components thereof. In addition, leaving free residual disinfectant in the fluid circuit may not be desired in certain situations or environments.

In various embodiments, a patient is not operatively connected to the thermal control unit during disinfection of the fluid circuit. For example, the patient may not be in the vicinity of, e.g. the same room as, the thermal control unit. This is useful for preventing inadvertent exposure of the patient to the disinfectant, aqueous mixture, free-chlorine, or residues thereof. In related embodiments, a patient therapy device is not operatively connected to the thermal control unit during disinfection of the fluid circuit. This is useful for reducing the amount of time required for disinfection, because many times, patient therapy devices are disposed of after use and there is no need for disinfection. However, it is to be appreciated that a patient therapy device may be operatively connected to the thermal control unit during disinfection of the fluid circuit, for example, in instances where the patient therapy device is reusable or merely to provide a closed-loop for the fluid circuit (e.g. to better facilitate circulation of the aqueous mixture).

In various embodiments, the method further comprises a rinsing portion. This is useful for rinsing the fluid circuit of the aqueous mixture or residue thereof. In these embodiments, the method further comprises the step of circulating water (e.g. fresh water) in the thermal control unit. The water may be as described above, e.g. distilled water. The method further comprises the step of removing the water from the thermal control unit to rinse the fluid circuit. These circulating and removing steps may be repeated at least once to further rinse the fluid circuit. In certain embodiments, the circulating and removing steps are repeated at least twice or at least three (or more) times to further rinse the fluid circuit.

In various embodiments, the method further comprises a maintenance portion. This is useful for substantially maintaining disinfection of the fluid circuit. Specifically, the fluid circuit can be used for a period of time, a certain number or uses, or stored for a period of time before the disinfection (or shock) portion of the method described above is repeated. In these embodiments, the aqueous mixture and the disinfectant of the shock portion of the method is further defined as a first aqueous mixture and a first disinfectant. The method further comprises the step of providing a second aqueous mixture different from the first aqueous mixture (although in some embodiments described below, the first and second aqueous mixtures may be substantially the same). The method yet further comprises the step of circulating the second aqueous mixture in the thermal control unit, and more specifically circulating in the fluid circuit of the thermal control unit. The second aqueous mixture may be provided and circulated in the same or a similar manner as that of the first aqueous mixture. Unlike generally with the shock portion, a patient and/or patient therapy device may be operatively connected to the thermal control unit during the maintenance portion of the method.

The second aqueous mixture comprises a second disinfectant. The second disinfectant may be the same as or different from the first disinfectant. For example, the second disinfectant may comprise free-chlorine (e.g. provided by a chlorinated isocyanurate), a phenol, H₂O₂, or combinations thereof. In certain embodiments, the second disinfectant comprises free-chlorine provided by NaDCC, thymol, H₂O₂ provided by sodium percarbonate, or combinations thereof. Other disinfectants understood in the art may also be utilized as the second disinfectant. If the second disinfectant is the same as the first disinfectant, the second disinfectant is generally present in the second aqueous mixture in an amount less than the amount of the first disinfectant present in the first aqueous mixture. This distinguishes the maintenance portion from the “shock” (or episodic) portion of the method. However, in embodiments where H₂O₂ is utilized, the amount of the H₂O₂ is generally the same or similar between the maintenance and shock portions of the method as further described below.

In various embodiments, the second disinfectant comprises free-chlorine. In certain embodiments, the free-chlorine is present in the second aqueous mixture in an amount less than 2,000, less than 1,500, less than 1,000, less than 500, or less than 100, ppm. In further embodiments, the free-chlorine is present in the second aqueous mixture in an amount of from about 0.5 ppm to about 95 ppm, alternatively in an amount of from about 1 ppm to about 75 ppm, alternatively in an amount of from about 1 ppm to about 50 ppm, alternatively in an amount of from about 2 ppm to about 25 ppm, alternatively in an amount of from about 2 ppm to about 10 ppm, or alternatively in an amount of from about 2 ppm to about 6.5 ppm. Various ranges and subranges of these amounts are also contemplated. In addition, these amounts can be adjusted to account for inclusion of the phenol and/or H₂O₂ (and/or other disinfectants) in embodiments including a combination of second disinfectants. It is to be appreciated that the amount of free-chlorine in the second aqueous mixture will gradually decrease with the passage of time. As such, the ppm amounts described above may be referred to as initial amounts, formed amounts, loaded amounts, or use/application amounts.

In various embodiments, the second disinfectant comprises the phenol. In certain embodiments, the phenol is present in the second aqueous mixture in an amount less than 13,000, less than 12,000, less than 11,000, or less than 10,000, ppm. In further embodiments, the phenol is present in the second aqueous mixture in an amount of from about 1,000 ppm to about 12,000 ppm, alternatively in an amount of from about 1,000 ppm to about 10,000 ppm, alternatively in an amount of from about 1,000 ppm to about 8,000 ppm, alternatively in an amount of from about 1,000 ppm to about 7,500 ppm, alternatively in an amount of from about 1,000 ppm to about 5,000 ppm, or alternatively in an amount of from about 1,500 ppm to about 2,500 ppm. Various ranges and subranges of these amounts are also contemplated. In addition, these amounts can be adjusted to account for inclusion of the free-chlorine and/or H₂O₂ (and/or other disinfectants) in embodiments including a combination of second disinfectants.

In various embodiments, the second disinfectant comprises H₂O₂. In certain embodiments, the H₂O₂ is present in the second aqueous mixture in an amount of at least about 5,000 ppm. In further embodiments, the H₂O₂ is present in the second aqueous mixture in an amount of from about 5,000 ppm to about 100,000 ppm, alternatively in an amount of from about 5,000 ppm to about 80,000 ppm, alternatively in an amount of from about 5,000 ppm to about 50,000 ppm, alternatively in an amount of from about 5,000 ppm to about 40,000, alternatively in an amount of from about 5,000 ppm to about 30,000 ppm, alternatively in an amount of from about 5,000 ppm to about 20,000 ppm, alternatively in an amount of from about 5,000 ppm to about 10,000 ppm, alternatively in an amount of about 5,000 ppm. Various ranges and subranges of these amounts are also contemplated. In addition, these amounts can be adjusted to account for inclusion of the free-chlorine and/or phenol (and/or other disinfectants) in embodiments including a combination of second disinfectants.

In various embodiments, the (first and/or second) aqueous mixture(s) is/are substantially free of other disinfectants, disinfectant generators, and/or free-chlorine provided by such disinfectants. Without being bound or limited to any particular theory, it is thought that these embodiments are useful for prolonging the working life of the fluid circuit and therefore the thermal control unit (e.g. by reducing material of construction attack/wear/breakdown), for promoting safer working conditions (e.g. by reducing the likelihood of irritation by handling or inhalation), and for promoting increased disinfection of the thermal control unit and surrounding area (e.g. by increasing the level of disinfection and/or by reducing the likelihood of bacterial growth and/or production of bacterial growth promotors, such as ammonia, nitrate, nitrite, etc.).

In these embodiments, the aqueous mixture(s) is/are substantially free of a bleach (e.g. sodium hypochlorite) and free-chlorine provided by a bleach. Alternatively or in addition, the aqueous mixture(s) is/are substantially free of a sulfonamide (e.g. tosylchloramide or “Chloramine-T”) and free-chlorine provided by a sulfonamide. Alternatively or in addition, the aqueous mixture(s) is/are substantially free of a quaternary ammonium compound (e.g. benzyl-C₁₂₋₁₈-alkyldimethyl, chlorides or AIRKEM A-33®) and free-chlorine provided by a quaternary ammonium compound. Alternatively or in addition, the aqueous mixture(s) is/are substantially free of a chloramine (e.g. monochloramine). If these disinfectants (and/or disinfectant generator) or related compounds are present, the level of such in the aqueous mixture(s) is typically less than about 10 ppm, alternatively less than about 5 ppm, or alternatively less than about 1 ppm. In various embodiments, the aqueous mixture(s) completely exclude(s) such disinfectants (and/or disinfectant generators) or related compounds.

In various embodiments, the disinfectant component is provided (or introduced) via at least one patient therapy device (and/or hose/line) connected to the thermal control unit. For example, NaDCC and/or sodium percarbonate (e.g. tablets, granules, and/or powder) can be present (and/or disposed) in a patient therapy device (e.g. a wrap, a pad, a blanket, etc.). The disinfectant component is then circulated in the fluid circuit when water is circulated in the thermal control unit during, for example, normal use where water flows to and from the patient therapy device. Utilizing “loaded” or “preloaded” patient therapy devices in this manner is useful for refreshing or maintaining disinfection of the fluid circuit.

As introduced above, the aqueous mixture of this disclosure has increased material compatibility relative to conventional disinfectants, such as bleach. The fluid circuit of the thermal control unit includes an inner surface. In many thermal control units, the inner surface comprises a material (i.e., a material of construction) selected from the group consisting of metallic materials, polymeric materials, and combinations thereof. In various embodiments, the material is selected from the group of steel and steel alloys, copper and copper alloys, rubbers, thermoplastics, or combinations thereof. In further embodiments, the material can be selected from stainless steel, copper, brass, and other alloys, ethylene propylene diene monomer (EPDM), acetyl, polypropylene and other rubbers and plastics, glass, etc. One of skill in the art appreciates that the material can vary depending on the type and/or location within the fluid circuit. This disclosure is not limited to a particular material of the fluid circuit.

In alternate embodiments, the maintenance portion of the method may utilize one or more of the “other” aforementioned disinfectants or disinfectant generators to form aqueous mixtures. In these embodiments, one or more of the following may be used in addition or alternate to the free-chlorine provided by the chlorinated isocyanurate, the phenol, and/or the H₂O₂: a bleach and free-chlorine provided by a bleach; a sulfonamide and free-chlorine provided by a sulfonamide; a quaternary ammonium compound and free-chlorine provided by a quaternary ammonium compound; a chloramine; and combinations thereof. One of skill in the art can readily calculate dosing depending on, for example, the particular source (e.g. purity or wt %) and desired ppm of disinfectant.

A system is also provided. The system comprises a thermal control unit having a fluid circuit for delivering temperature controlled fluid to at least one patient therapy device. An aqueous mixture is disposed in the fluid circuit. The aqueous mixture is as described above for the method, i.e., it includes the disinfectant. The fluid circuit of the thermal control unit is also as described above for the method. The method can be used to disinfect the system. Additional embodiments of the system are described below.

Suitable thermal control units or systems for this disclosure are (commercially) available from various companies, such as from STRYKER®, from Cincinnati Sub-Zero (CSZ), from Sorin Group, and from C.R. Bard, Inc. (BARD MEDICAL). Specific examples of thermal control units or systems (and related components, e.g. patient therapy devices) include those commercially available from STRYKER® under the trade name ALTRIX™ and MEDI-THERM®, such as ALTRIX™ Precision Temperature Management Systems and MEDI-THERM® III Hyper/Hypothermia Machines; from CSZ under the trade name BLANKETROL® and HEMOTHERM®, such as BLANKETROL® III hyper-hypothermia systems and HEMOTHERM® CE cardiovascular heater/cooler systems; from Sorin Group, such as Sorin 3T Heater-Cooler Systems; and from C.R. Bard, Inc. under the trade name ARCTIC SUN®, such as ARCTIC SUN® 5000 systems. Further examples of suitable thermal control units, systems, apparatuses or related components, are disclosed in U.S. Pat. No. 6,517,510 (the ′510 patent); U.S. Pat. No. 6,692,518; U.S. Pat. No. 6,818,012; U.S. Pat. No. 7,044,960; U.S. Pat. No. 8,491,644; and U.S. Pat. No. 8,647,374; and in U.S. Patent App. Pub. No. 2014/0343639 (the ′639 publication); each of which is incorporated herein by reference in its entirety.

In various embodiments, a thermal control unit is provided that is adapted to deliver temperature controlled fluid to a patient. The thermal control unit includes a plurality of outlets adapted to fluidly connect to a plurality of patient therapy devices, such as, but not limited to, one or more thermal pads, blankets, wraps, vests, boots, socks, caps, or the like. Patient therapy devices may also be referred to as thermal transfer devices. The outlets are adapted to deliver the temperature controlled fluid to the patient therapy devices when the patient therapy devices are connected thereto. The thermal control unit includes a sensing subsystem to monitor the connection status of the outlets and/or the utilization of the fluid circuit(s) defined between the thermal control unit and the patient therapy device(s). The thermal control unit further includes an indicator adapted to provide an indication to a user if a patient therapy device is added to, or removed from, any one or more of the outlets while the control unit is delivering the temperature controlled fluid to the patient.

Referring now to the Figures, wherein like numerals generally indicate like parts throughout the several views, a thermal control unit is shown generally at 22. In various embodiments, the fluid circuit (generally indicated by arrows 56) of the thermal control unit 22 comprises a circulation channel 54 for holding a fluid (e.g. water or the aqueous mixture; not shown). A pump 52 is in fluid communication with the circulation channel 54 for circulating the fluid. At least one outlet 24 is in fluid communication with the circulation channel 54 for sending fluid to at least one patient therapy device 32, e.g. a thermal pad 32a. At least one inlet 26 is in fluid communication with the circulation channel 54 for receiving fluid from the patient therapy device(s) 32.

An outlet manifold 60 can define one or more outlets 24 (or outlet ports 24). Likewise, an inlet manifold 62 can define one or more inlets 26 (or inlet ports 26). At least one bypass line 62 may be in fluid communication with the outlet 24 (or outlet manifold 60) and the inlet 26 (or inlet manifold 64) for allowing circulation of the fluid in the absence of the patient therapy device(s) 32. The manifolds 60, 64 are useful for providing multiple ports 24, 26, which in turn may be used for multiple patient therapy devices 32. In various embodiments, there is at least one bypass line 62 (or 62a) which is internally located between the manifolds 60, 64. Optionally, at least one supply line 30 is in fluid communication with the outlet 24 (or outlet manifold 60) for sending fluid to the patient therapy devices(s) 32. Optionally, at least one supply line 30 is in fluid communication with the inlet 26 (or inlet manifold 64) for receiving fluid from the patient therapy devices(s) 32. Alternatively, such supply lines may be part of the patient therapy devices(s) 32.

A heat exchanger 58 is operatively connected to the fluid circuit 56 for heating and/or cooling the fluid in the fluid circuit 56. A reservoir 38 is in fluid communication with the fluid circuit 56 for providing fluid to the fluid circuit 56. The reservoir 38 may be removable or fixed. In addition, the reservoir 38 may be open (generally as shown) or closed. In certain embodiments, the fluid circuit 56 is completely closed from the atmosphere, whereas in other embodiments, the fluid circuit 56, at one or more points, is open to the atmosphere. Optionally, a separator 68 is in fluid communication with the circulation channel 54 for separating entrained air from the fluid. If overflowed, the separator 68 may dump fluid into the reservoir 38. Optionally, a filter 66 is in fluid communication with the circulation channel 54 for filtering the fluid. In general, a controller 72 is in electrical communication with at least one of the pump 52 and the heat exchanger 58 for controlling flow and/or temperature of the fluid in the fluid circuit 56. The controller 72 may be connected to other components as well, such as I/O devices, e.g. graphical user interface (GUI), keypad, screen, etc.

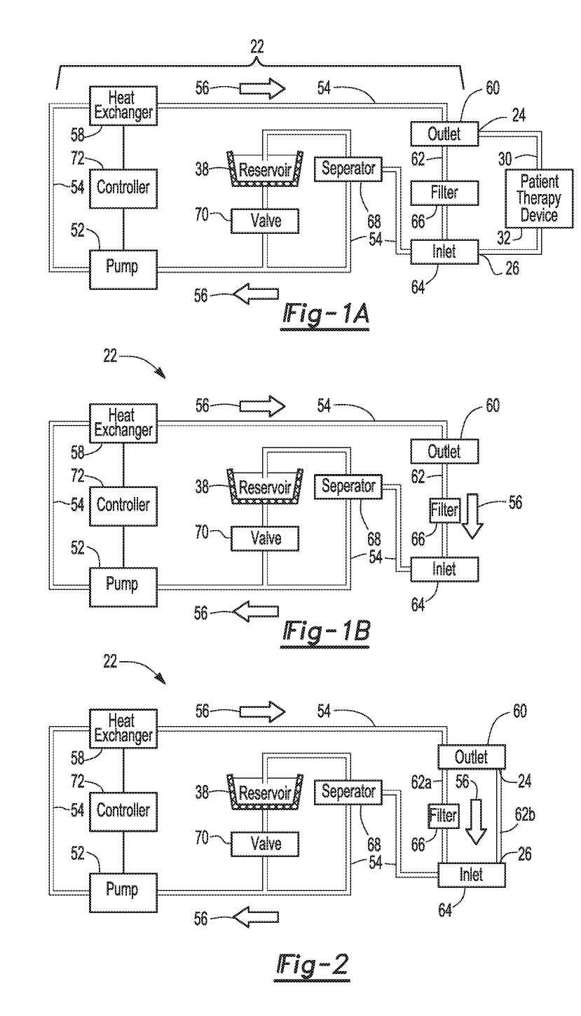

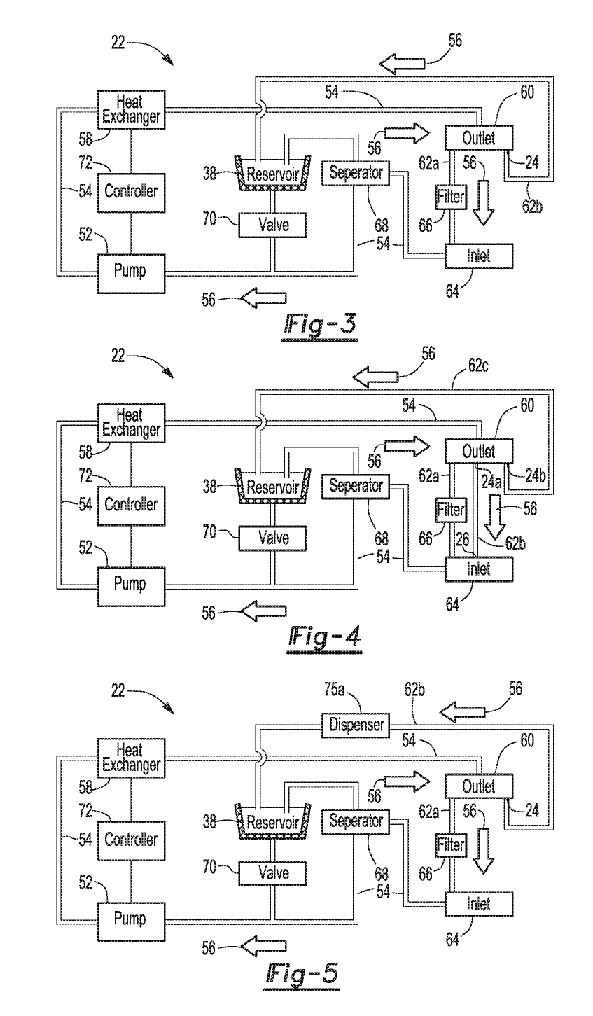

Referring to FIG. 1A, the patient therapy device 32 is connected between outlet 24, 60 and inlet 26, 64. The patient therapy device 32 is removed in FIG. 1B. In FIG. 2, bypass line 62b is connected between outlet 24, 60 and inlet 26, 64. This is useful for disinfecting the ports 24, 26. In FIG. 3, first bypass line 62a is connected between outlet 24, 60 and inlet 26, 64 and second bypass line 62b is routed from outlet 24, 60 to reservoir 38. This is useful for disinfecting the reservoir 38. In FIG. 4, first bypass line 62c is routed from outlet 24, 60 to reservoir 38, whereas second bypass line 62b and third bypass line 62a are connected between outlet 24, 60 and inlet 26, 64.

Referring to FIGS. 5 to 8, the thermal control unit 22 further comprises a dispenser 75. The dispenser 75 is useful for providing the disinfectant component and/or the aqueous mixture comprising the disinfectant. The dispenser 75 can be of various designs and/or be at various locations in the fluid circuit 56 or the thermal control unit 22. The dispenser 75 may also be located outside of the thermal control unit 22, e.g. attached in place of patient therapy device 32. In certain embodiments, the dispenser 75 mimics the dispenser disclosed in co-pending U.S. Provisional Patent Application No. 62/406,676 (Atty. Docket No. 143667.169707 (P-548)), which is incorporated herein by reference in its entirety.

In various embodiments, the dispenser 75 is configured such that the disinfectant component and/or the aqueous mixture can be added to the reservoir 38 whenever the reservoir 38 is refilled with fresh/new water. It may be included in the GUI that whenever a user adds water to the reservoir 38, they are reminded to check on the dispenser 75 and/or add the disinfectant component and/or the aqueous mixture to the dispenser 75 or to the reservoir 38. The dispenser 75 is not required for the system, but can help automate or facilitate the disinfection method of this disclosure. In other embodiments, the dispenser 75 is configured in such a manner as to add the disinfectant component automatically after a set period of time to provide disinfection of the system without active input from the user.

In certain embodiments, the dispenser 75 mimics a chlorination device as disclosed in U.S. Pat. No. 9,102,557 (the ′557 patent), which is incorporated herein by reference in its entirety. FIG. 1 of the ′557 patent illustrates an embodiment of the chlorination device that may be utilized as the dispenser 75 of the subject invention. Chlorination devices of this type are commercially available from Medentech Ltd. under the trade name FLOGENIC®. Similarly designed dispensers, e.g. such as those mimicking erosion feeders common in the pool/spa art, may also be utilized as the dispenser 75. For example, the dispenser 75 can hold one or more tablets and/or granules of the disinfectant component and be configured and connected to the fluid circuit 56 such that water flow contacts and erodes/dissolves the disinfectant component (e.g. NaDCC) into solution to form the aqueous mixture.

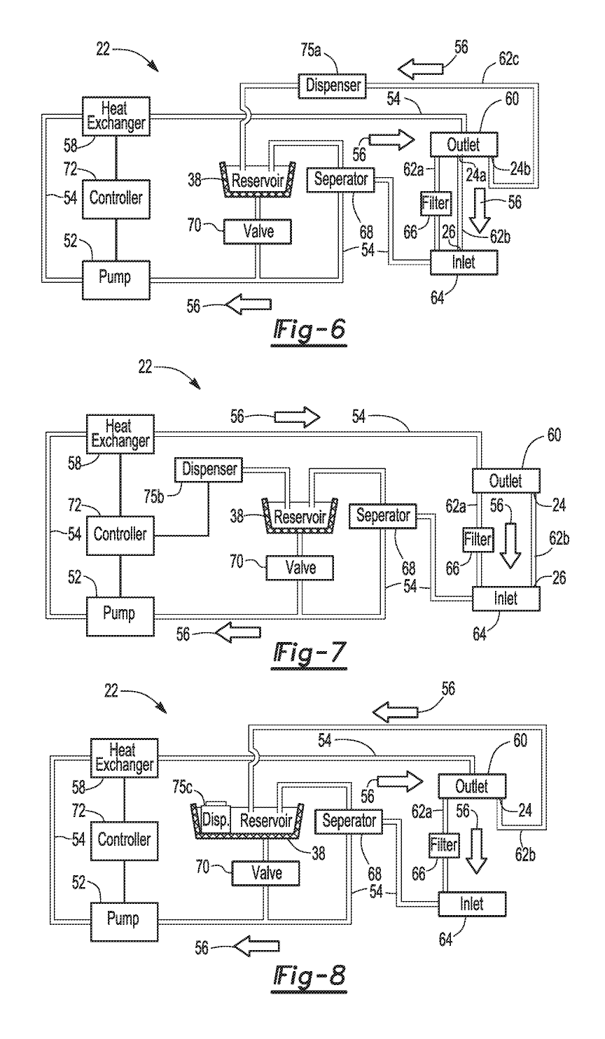

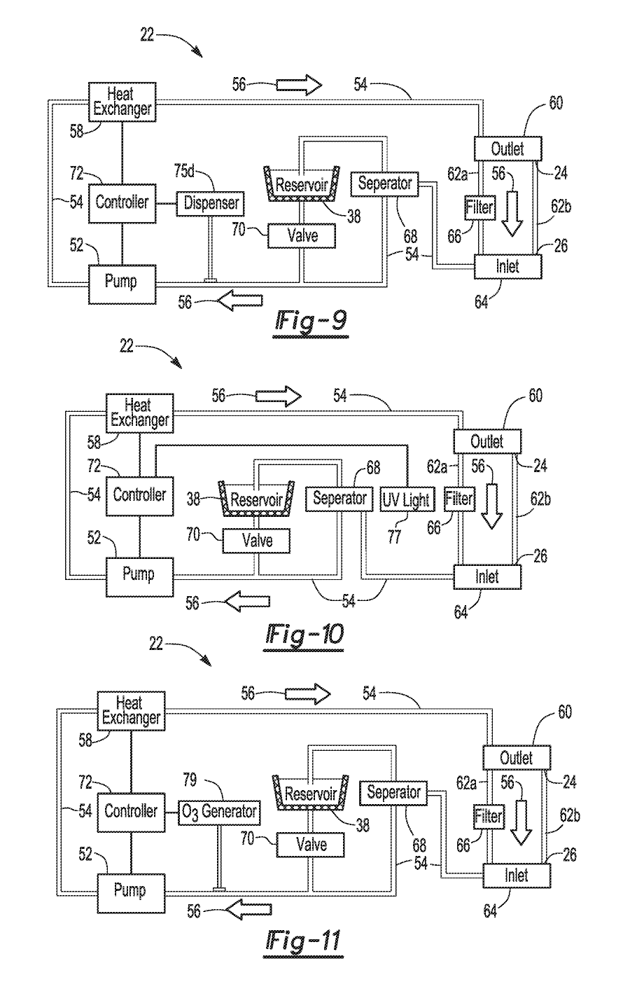

In FIG. 5, bypass line 62b is routed from outlet 24, 60 to dispenser 75a. The dispenser 75a may be as described above. While dispenser 75a is shown as being routed to reservoir 38, alternatively dispenser 75a may be routed to inlet 26, 64. In FIG. 6, first bypass line 62c is routed from outlet 24, 60 to dispenser 75a. The dispenser 75a is routed to reservoir 38 and second bypass line 62b is connected between outlet 24a, 60 and inlet 26, 64. In FIG. 7, bypass line 62b is connected between outlet 24, 60 and inlet 26, 64. Dispenser 75b is routed to reservoir 38. The dispenser 75b may be configured to engage reservoir 38 to supply the disinfectant component and/or the aqueous mixture to reservoir 38. In FIG. 9, dispenser 75d can be connected directly to circulation channel 54. The dispenser 75d may be configured to engage circulation channel 54 to supply the disinfectant component and/or the aqueous mixture to circulation channel 54. Controller 72 may be used to activate or at least monitor dispenser 75b, 75d.

In FIG. 8, bypass line 62b is routed from outlet 24, 60 to reservoir 38. Dispenser 75c is at least partially disposed in reservoir 38. Dispenser 75c can be fully or partially submerged when reservoir 38 is full of fluid (e.g. water). The dispenser 75c can hold one or more tablets and/or granules of the disinfectant component and be configured such that water in or directed to reservoir 38 contacts and erodes/dissolves the disinfectant component (e.g. NaDCC and/or sodium percarbonate) into solution to form the aqueous mixture. In various embodiments, dispenser 75c mimics a basket filter (or strainer) and can be of various screen or mesh size to control particle size of the disinfectant component while dissolving into solution. This helps to prevent large particles or agglomerations of the disinfectant component from potentially plugging or blocking components of the system, e.g. valve 70 or pump 52. While shown hanging from the side of reservoir 38, dispenser 75c may merely sit or even float in reservoir 38 (e.g. similar to a tea leaf infuser or floating erosion feeder used in pools).

In various embodiments, the thermal control unit 22 further comprises an ultraviolet (UV) light 77. The UV light emits UV radiation when powered. In FIG. 10, the UV light 77 is adjacent separator 68. This location is useful because fluid (e.g. water) collects in separator 68 after returning from patient therapy devices(s) 32 or bypass line 62 before returning back to pump 52. While not shown, the UV light 77 may also be situated in other locations, such as after the heat exchanger 58 and before the outlet 62 or just before the pump 52 to maximize exposure. In many embodiments, tubing and/or other aspects of the circulation channel 54 is relatively clear. In certain embodiments, UV light 77 would be in operation while thermal control unit 22 is powered. The UV light 77 can be adjusted to an appropriate power or intensity, e.g. via controller 72, to account for the volume and flow rate of fluid in separator 68.

While not shown, one or more shields can be added around UV light 77 to ensure that no UV radiation escapes a localized area or thermal control unit 22. The UV light 77 can be of various configurations. In certain embodiments, the UV light 77 mimics the UV device disclosed in U.S. Pat. No. 8,460,353, which is incorporated herein by reference in its entirety. As understood in the art, UV light is very effective at inactivating certain pathogens in low turbidity water. One drawback is that UV light's disinfection effectiveness decreases as turbidity increases, a result of the absorption, scattering, and shadowing caused by suspended solids. However, this scenario is generally not a problem in thermal control units 22. Perhaps a more notable disadvantage to the use of UV radiation is that it leaves no residual disinfectant in the water, i.e., it must always be present to function. Therefore, utilizing the aqueous mixture of this disclosure as a primary disinfectant and UV radiation as an optional secondary disinfectant may be useful for certain disinfection regimes. In other embodiments, UV radiation is the primary disinfectant.

In various embodiments, the thermal control unit 22 further comprises an ozone (O₃) generator 79. The ozone generator 79 emits ozone when powered. In FIG. 11, the ozone generator 79 is connected to circulation channel 54. Infusing or injecting ozone directly into the fluid path (e.g. by bubble contact) is useful for keeping ozone from escaping thermal control unit 22. In general, ozone should be introduced downstream of reservoir 38 and separator 68 to prevent inadvertent or excessive escape of ozone from the system. In certain embodiments, ozone generator 79 would be in operation while thermal control unit 22 is powered. The ozone generator 79 can be adjusted to an appropriate power or intensity, e.g. via controller 72, to account for the volume and flow rate of fluid in circulation channel 54.

The ozone generator 79 can be of various configurations. For example, ozone generator 79 can be configured to produce ozone by passing oxygen through UV light or a “cold” electrical discharge. As understood in the art, ozone is an unstable molecule which readily gives up one atom of oxygen providing a powerful oxidizing agent which is toxic to most waterborne pathogens. Ozone is a very strong, broad spectrum disinfectant. One drawback to the use of ozone is that it leaves no residual disinfectant in the water, i.e., it must always be present to function. Therefore, utilizing the aqueous mixture of this disclosure as a primary disinfectant and ozone as an optional secondary disinfectant may be useful for certain disinfection regimes. In other embodiments, ozone is the primary disinfectant.

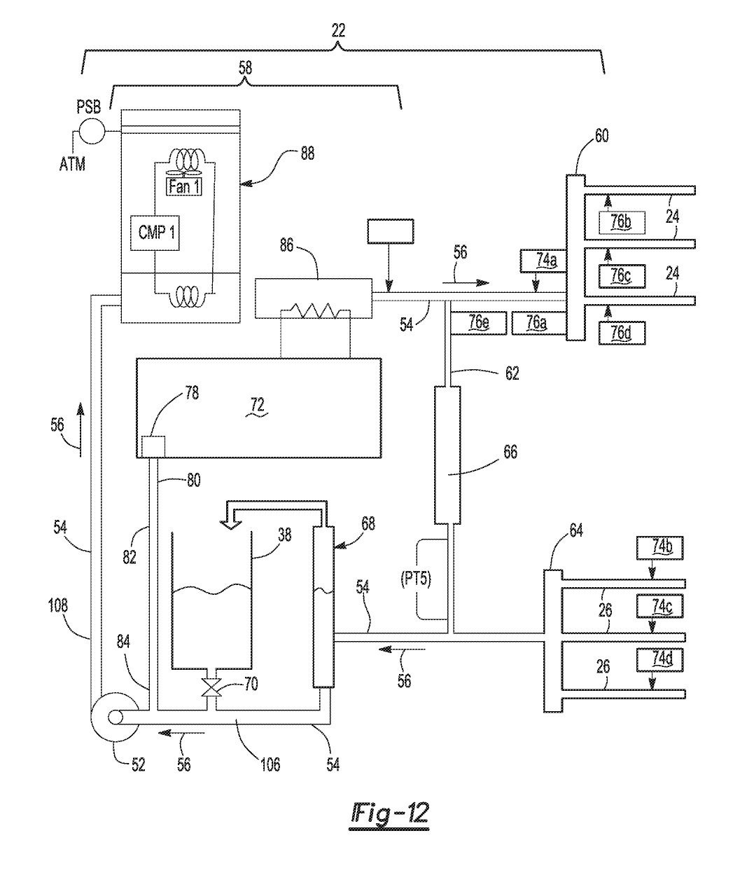

In various embodiments, the system of this disclosure is selected from the thermal control systems disclosed in U.S. Patent App. Pub. No. 2014/0343639 (the ′639 publication), which is incorporated herein by reference in its entirety. FIG. 12 illustrates one such system, with the numerals utilized below generally being the same as those in the ′639 publication.

FIG. 12 illustrates a diagram of the internal construction of thermal control unit 22. As seen in FIG. 12, thermal control unit 22 includes a pump 52 for circulating fluid through a circulation channel 54. Pump 52, when activated, circulates the fluid through circulation channel 54 in the direction of arrows 56 (clockwise in FIG. 12). Starting at pump 52, the circulating fluid first passes through a heat exchanger 58 where it is delivered to an outlet manifold 60 having the plurality of outlet ports 24. A bypass line 62 is fluidly coupled to outlet manifold 60 and an inlet manifold 64. Bypass line 62 allows fluid to circulate through circulation channel 54 even in the absence of any thermal pads 32a or lines 30 being coupled to any of outlet and inlet ports 24 and 26. In the illustrated embodiment, bypass line 62 includes an optional filter 66 that is adapted to filter the circulating fluid. If included, filter 66 may be a particle filter adapted to filter out particles within the circulating fluid that exceed a size threshold, or filter 66 may be a biological filter adapted to purify or sanitize the circulating fluid, or it may be a combination of both.

Inlet manifold 64 includes the plurality of inlet ports 26 that receive fluid returning from the one or more connected thermal pads 32a. The incoming fluid from inlet ports 26, as well as the fluid passing through bypass line 62, travels back toward the pump 52 into an air separator 68. Air separator 68 includes a generally vertical tube that is open at its top end to atmospheric pressure. Any air bubbles that are entrained in the circulating fluid will naturally rise up through air separator 68 and be vented to the atmosphere. After passing through air separator 68, the circulating fluid flows past a valve 70 positioned beneath fluid reservoir 38 and back to pump 52 via pump inlet tube 106. The circulating fluid exits pump 52 via pump outlet tube 108. Some to all of the circulating fluid may also bypass the air separator 68 and/or valve 70 when returning to the pump 52, alternatively some to all of the circulating fluid may also bypass the air separator 68 when returning to the pump 52.

Thermal control unit 22 further includes controller 72 that is contained within main body 36 and in electrical communication with a variety of different sensors and/or actuators. More specifically, controller 72 is in electrical communication with pump 52, heat exchanger 58, and control panel 46. While not illustrated in FIG. 12, controller 72 is further in communication with first, second, third, and fourth temperature sensors 74a, b, c, and d, respectively, as well as with first, second, third, fourth, and fifth pressure sensors 76a, b, c, d, and e, respectively (or turbine flow sensors, if used, as discussed below). Controller 72 is also in communication with an air pressure sensor 78 that is positioned in gaseous communication with a top end 80 of a level sensing tube 82. Level sensing tube 82 is generally vertical and includes a lower end 84 that is in fluid communication with fluid circulation channel 54.

Controller 72 includes any and all electrical circuitry and components necessary to carry out the functions and algorithms described in the ′639 publication, as would be understood by one of ordinary skill in the art. Generally speaking, controller 72 may include one or more microcontrollers, microprocessors, and/or other programmable electronics that are programmed to carry out the functions described in the ′639 publication. It will be understood that controller 72 may also include other electronic components that are programmed to carry out the functions described in the ′639 publication, or that support the microcontrollers, microprocessors, and/or other electronics. The other electronic components include, but are not limited to, one or more field programmable gate arrays, systems on a chip, volatile or nonvolatile memory, discrete circuitry, integrated circuits, application specific integrated circuits (ASICs) and/or other hardware, software, or firmware, as would be understood by one of ordinary skill in the art. Such components can be physically configured in any suitable manner, such as by mounting them to one or more circuit boards, or arranging them in other manners, whether combined into a single unit or distributed across multiple units. Such components may be physically distributed in different positions in thermal control unit 22, or they may reside in a common location within thermal control unit 22. When physically distributed, the components may communicate using any suitable serial or parallel communication protocol, such as, but not limited to, CAN, LIN, Firewire, I-squared-C, RS-232, RS-485, universal serial bus (USB), etc.

As illustrated in FIG. 12, heat exchanger 58 includes both a heater 86 and a chiller 88. Heat exchanger 58 is therefore capable of both cooling the circulating liquid and heating the circulating liquid. In some instances, where precise temperature control is desired, such heating and cooling may occur at the same time. That is, the circulating fluid may be sequentially both heated and cooled, with the latter heating or cooling occurring if the first temperature adjustment overshoots the intended target temperature. In other embodiments, heat exchanger 58 may include only a chiller 88 or only a heater 86, depending upon the desired type of temperature control. In the illustrated embodiment where heat exchanger 58 includes both a chiller 88 and a heater 86, both heater 86 and chiller 88 are in communication with, and under the control of, controller 72.

Controller 72 uses the outputs of temperature sensors 74a, b, c, and d to control the temperature of the circulating fluid. That is, controller 72 uses the outputs of temperature sensors 74a, b, c, and d to control heat exchanger 58 such that the fluid circulating therethrough has its temperature adjusted (or maintained) in accordance with the operating mode (manual or automatic) selected by the user of thermal control unit 22. In one embodiment, controller 72 controls the temperature of the circulating fluid by using both an output temperature value (as measured by temperature sensor 74a) and a return temperature value (as determined from a mathematical combination of the readings from sensors 74b, c, and/or d). More specifically, controller 72 averages the temperature readings from sensors 74b, c, and d (or a subset of these three sensors if fewer than all three return ports 26 are being utilized) to generate the return temperature value. Controller 72 uses the return temperature value as the measured variable in implementing a closed loop proportional-integral (PI) controller for controlling the circulating fluid temperature. The target temperature of the circulating fluid is supplied either by a user (manual mode) or automatically by controller 72 (in automatic mode) based on a desired patient temperature and the current patient temperature (as determined from one of probes 34). Controller 72 thus compares the measured return temperature value to the target temperature and, if different, makes corresponding adjustments in the temperature (via heat exchanger 58) in order to change the current temperature to the target temperature. When carrying out this control using the PI controller, controller 72, in one embodiment, uses the output temperature value from temperature sensor 74a to adjust the limits of integration of the PI controller. Other types of controllers may be used in other embodiments for adjusting the temperature of the circulating fluid.

Controller 72 is further configured to display each of the temperatures sensed by temperature sensors 74b, 74c, and 74d. That is, controller 72 is configured to display to the user the individual temperature readings associated with the fluid returning to each of the inlet ports 26. Because each inlet port 26 may be attached to a different thermal pad 32a, which in turn is likely positioned at a different location on the patient's body, the returning fluid from each thermal pad 32a may be at a different temperature. Further, it may be useful for a caregiver to know which of the multiple thermal pads 32a is responsible for the largest, or smallest, temperature change relative to the temperature of the outgoing fluid, and thereby the largest or smallest amount of heat transfer with respect to the patient. Thermal control unit 22 therefore provides the user with individualized temperature information for each of the multiple inlet ports. Further, controller 72 is configurable to also display the outgoing fluid temperature on control panel 46, as sensed by outgoing fluid temperature sensor 74a.

Controller 72 utilizes the data outputs from fluid pressure sensors 76a, b, c, d, and e in order to determine the flow rate or amount of flow volume. As would be understood by one of ordinary skill in the art, the flow volumes can be calculated based upon the difference in pressures between pressure sensor 76a and each of the outgoing pressure sensors 76b, 76c, and 76d (and/or the bypass pressure sensor 76e) as well as the known orifice sizes of the outlet ports 24 (and/or the bypass line 62). More specifically, controller 72 is configured to individually calculate the flow rate of fluid exiting out each of the three outlet ports 24, as well as the flow rate of fluid passing through bypass line 62. Controller 72 calculates the flow rate through a first outlet port 24 by using the difference in pressure between pressure sensor 76a and 76b (as well as other data, such as orifice sizes). Controller 72 calculates the flow rate through a second outlet port 24 by using the difference in pressure between pressure sensor 76a and 76c (as well as other data). And controller 72 calculates the flow rate through a third outlet port 24 by using the difference in pressure between pressure sensor 76a and 76d (as well as other data). Still further, controller 72 calculates the flow rate through the bypass line 62 by using the difference in pressure between pressure sensor 76a and 76e (as well as other data). Controller 72 is also configured to display each of the individual outlet port flow volumes on control panel 46 so that a user of control unit 22 will know the amount of fluid flowing to each individual thermal pad 32a. In some embodiments, controller 72 is also configured to display the amount of fluid flowing through bypass line 62 as well.

Controller 72 uses the flow data in its closed-loop feedback control of heat exchanger 58. In one embodiment, controller 72 uses a proportional-integral control loop (PI control). In other embodiments, controller 72 can be adapted to use a proportional-integral-derivative control loop (PID control). In still other embodiments, controller 72 may simply use proportional control with no integral or derivative terms. Regardless of the specific type of control loop used, controller 72 uses the information from the pressure sensors 76a-e, as well as the temperature sensors 74a-d in determining the control commands that are issued to heat exchanger 58.

In other embodiments, pressure sensors 76a, b, c, d, and/or e are replaced by turbine sensors that directly measure flow rates. Still further, in other embodiments, the positions of pressure sensors 76a, b, c, d, and/or e (or turbine flow sensors, if used) are changed from that shown in FIG. 12. For example, in one embodiment, outlet manifold pressure sensor 76a is replaced with a turbine flow sensor positioned just downstream of pump 52. In still another embodiment, pressure sensors 76b, c, and d (whether implemented as pressure sensors or turbine flow sensors) are positioned at inlet ports 26 rather than outlet ports 24. Still other variations are possible. In still another embodiment, both pressure sensors 76 and turbine flow sensors are used to measure fluid flow rates.

Removable reservoir 38 includes on its bottom a valve 71 (shown in FIG. 22 of the ′639 publication) that automatically cooperates with valve 70 within control unit 22 when reservoir 38 is inserted (into the position shown in FIGS. 2 and 22 of the ′639 publication). More specifically, valve 71 automatically closes when reservoir 38 is removed from control unit 22 so that any fluid that is contained within it, or that is added to it, will not leak out of reservoir 38. Likewise, valve 70 automatically closes when reservoir 38 is lifted out of control unit 22 so that any fluid in the control unit 22 does not leak out of it. When removable reservoir 38 is inserted into control unit 22, both valve 70 and valve 71 cooperate with each other to both open. This automatic opening allows fluid to flow either into or out of control unit 22, depending upon what fluid, if any, is already present within control unit 22 and the relative pressure of that fluid compared to any fluid that is contained within reservoir 38. Valves 70 and 71 may be commercially available valves, such as are available from Colder Products Company of St. Paul, Minn., or from other suppliers.

Control unit 22 is configured such that removable reservoir 38 can be removed while thermal therapy is being delivered to a patient without any interruption in that thermal therapy. That is, controller 72 will continue to control the delivery of temperature controlled fluid to one or more thermal pads 32a even if reservoir 38 is removed from unit 22. Controller 72 will provide an indication to a user that reservoir 38 has been removed (via a sensor discussed below), but this will not interrupt the delivery of temperature controlled fluid to a patient via pads 32a. In this manner, reservoir 38 can be removed and carried to a sink or other location for adding or draining water, or other fluid, to reservoir 38 simultaneously with the delivery of thermal therapy to a patient. If reservoir 38 is inserted back into control unit 22 during this delivery of thermal therapy to the patient, the reservoir valve 71 and valve 70 will automatically open and whatever fluid within reservoir 38, if any, will be put in fluid communication with the fluid circulating through control unit 22.

When reservoir 38 is first filled and control unit 22 is used for the very first time, the coupling of reservoir 38 to control unit 22 will cause the reservoir valve 71 and valve 70 to both open, as noted, thereby allowing the fluid within reservoir 38 to flow out and into a portion of circulating channel 54. More specifically, fluid will flow into pump 52, a portion of level sensing tube 82, and a portion of air separator 68. In the illustrated embodiment, the fluid will not flow into either outlet manifold 60 or inlet manifold 64 as those are positioned at a higher elevation than fluid reservoir 38 within control unit 22. Only when pump 52 is activated will fluid be pumped to these manifolds 60 and 64.

When pump 52 is activated, it will pump fluid throughout circulating channel 54 and any connected thermal pads 32a. The fluid needed to fill the spaces in circulating channel 54 and thermal pads 32a that were previously occupied by air is drawn from reservoir 38. Once the entire system (circulating channel 54, manifolds 60 and 64, and any connected pads 32a) is filled with fluid drawn from reservoir 38, any remaining fluid within reservoir 38 will remain within reservoir 38 and be substantially outside of the circulating loop of fluid. That is, the fluid within reservoir 38 will be substantially isolated from the circulating fluid such that temperature changes made to the circulating fluid will have little to no impact on the temperature of the fluid within reservoir 38. In this manner, it is not necessary to expend the extra time and energy that would otherwise be necessary to bring the volume of fluid within reservoir 38 to the desired temperature. Instead, any temperature adjustments made to the fluid are made only to the portion of the fluid that is circulating, thereby avoiding unnecessary expenditures of energy and time on heating or cooling fluid that does not circulate to the thermal pads 32a. In this manner, thermal control unit 22 operates as a tank-less thermal control unit that has a faster response time than many prior art thermal control units. That is, thermal control unit 22 is able to bring the circulating fluid to a desired temperature quicker and/or with less energy than thermal control units that include a tank and greater amounts of fluid within the thermally controlled circuit.

When pump 52 is deactivated after having been activated, the fluid within circulating channel 54 will drain downward due to gravity into the lower regions of circulating channel 54, as well as partially returning into reservoir 38, when attached. Any fluid within thermal pads 32a will also return to the lower regions of circulating channel 54 provided the thermal pads 32a are positioned at a height that is greater than the height of inlet ports 26 so that gravity may pull the fluid downward out of the thermal pads 32a and through inlet ports 26. The deactivation of the pump 52 will therefore return a portion of the circulating fluid to reservoir 38 while leaving another portion of the circulating fluid in the bottom areas of circulating channel 54. In order to more completely remove the fluid from circulating channel 54, a drain 92 (shown in FIG. 6 of the ′639 publication) can be opened to further drain the fluid out of control unit 22, if desired, as will be discussed in greater detail below.

Thermal control unit 22 is further in electrical communication with a reservoir sensor 90 (shown in FIG. 10 of the ′639 publication) that is adapted to electrically detect the presence or absence of reservoir 38. Reservoir sensor 90 may be any suitable sensor for detecting the absence and presence of reservoir 38. In the illustrated embodiment, reservoir sensor 90 is a Reed switch that is adapted to detect the absence or presence of a magnet (not shown) integrated into the bottom of the reservoir 38 at a location that aligns with sensor 90 (when reservoir 38 is coupled to unit 22). Reservoir sensor 90 communicates the presence or absence of reservoir 38 to controller 72 which, in turn, is configured to display that information on control panel 46, as well as to issue alerts or warnings if the user attempts to implement a function that is dependent upon the presence of reservoir 38 and sensor 90 is detecting its absence.

In FIG. 12, it is to be appreciated that the thermal pads 32a may of be replaced with other patient therapy devices 32. In addition, one of more bypass lines 62 may be used in place of the thermal pads 32a (or other patient therapy devices 32).

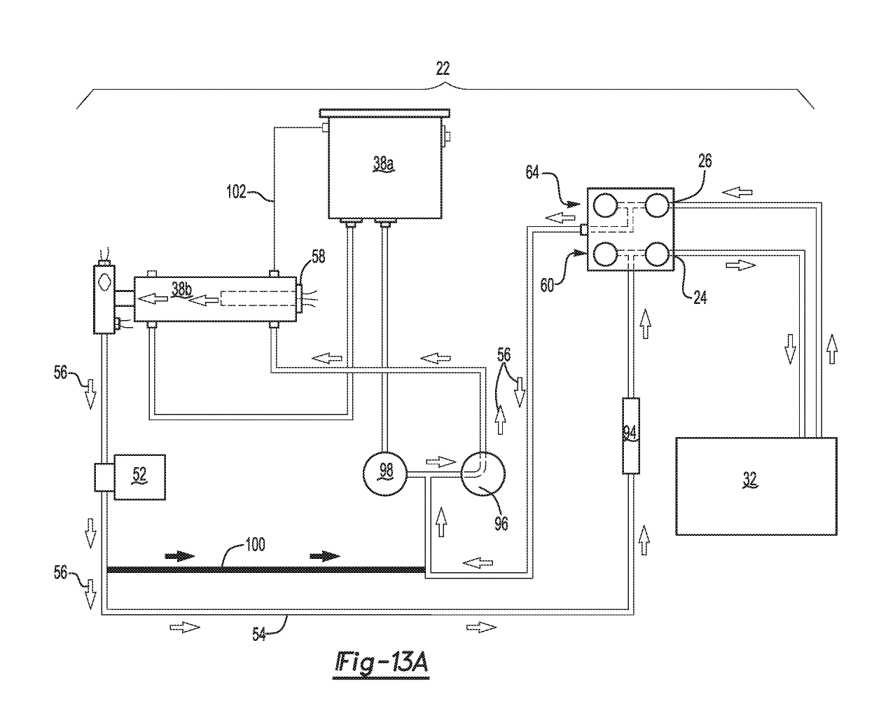

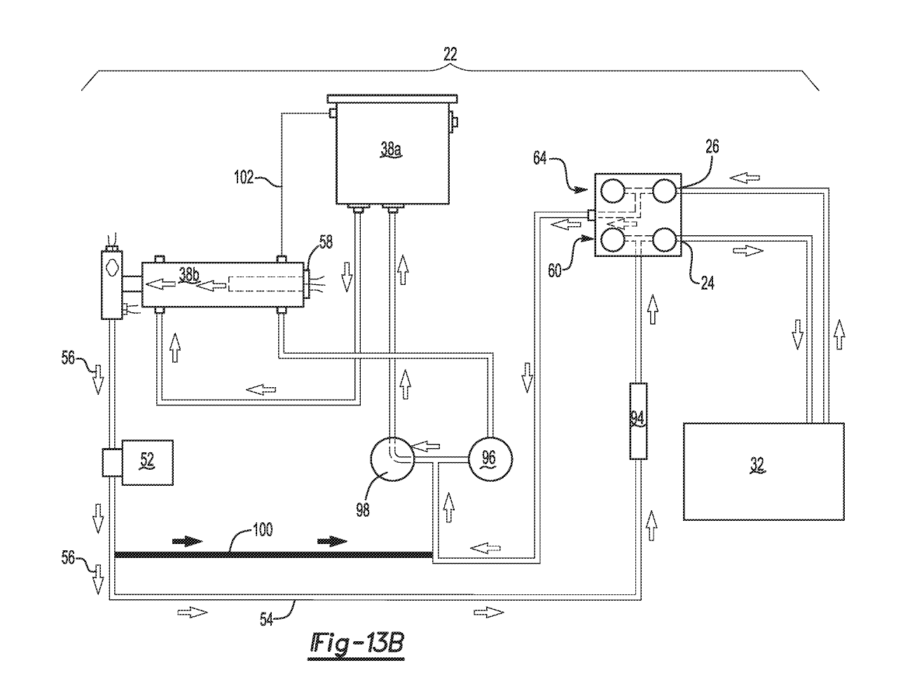

In various embodiments, the system of this disclosure is selected from the thermal control systems disclosed in U.S. Pat. No. 6,517,510 (the ′510 patent), which is incorporated herein by reference in its entirety. FIG. 4 of the ′510 patent illustrates a system that may be disinfected via the method of this disclosure. FIGS. 13A and 13B also illustrate one such system.

In FIG. 13A, the thermal control unit 22 is running in a heating mode, whereas in FIG. 13B, the thermal control unit 22 is running in a cooling mode. Again, active heating or cooling is not required for the method. In FIG. 13, the thermal control unit 22 includes a cold reservoir 38a and a hot reservoir 38b, with an air vent 120 connected therebetween. The disinfectant can be added (directly and/or indirectly) to either one or both of these reservoirs 38a, b. Hot solenoid 96 and cold solenoid 98 can be opened or closed, respectively, to change over from heating mode to cooling mode. A bypass 100 is available should flow switch 94 be closed or partially closed during operation.

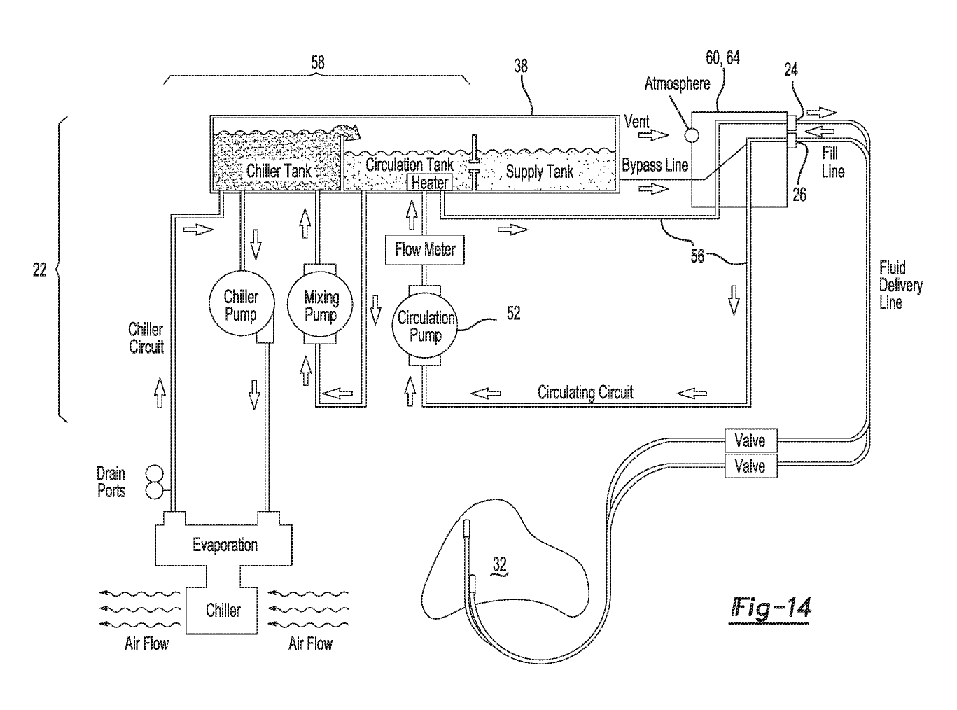

Another system that may be disinfected via the method of this disclosure is illustrated in FIG. 14. The thermal control unit 22 uses an alternate tank design (e.g. chiller, circulation and supply tanks) for cooling fluid. Specifically, the thermal control unit 22 includes a reservoir 38 having multiple zones. The disinfectant can be added (directly and/or indirectly) to one or more of these zones.

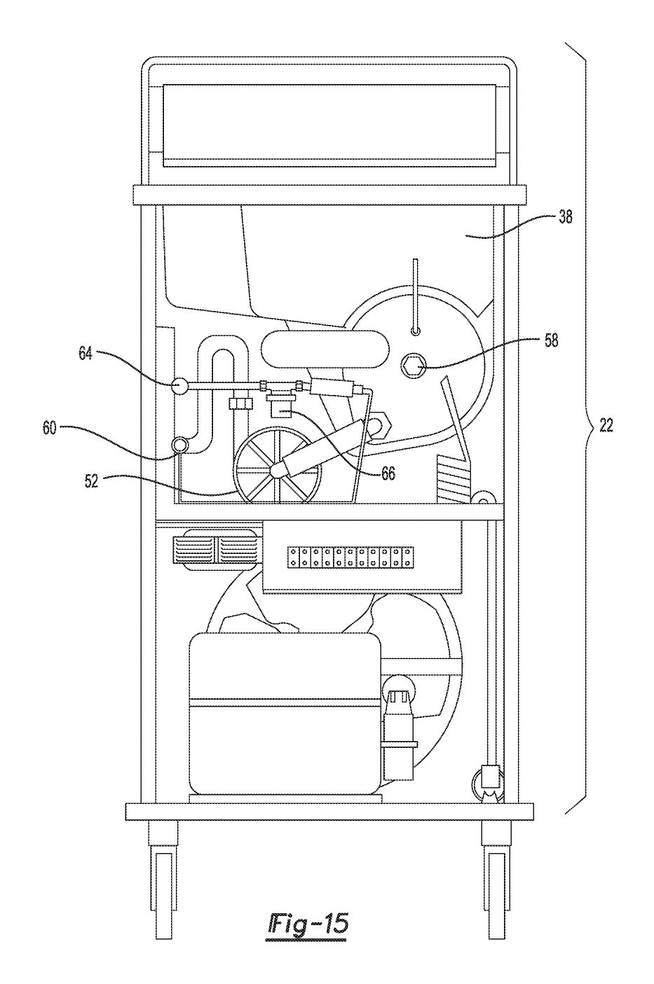

Yet another system that may be disinfected via the method of this disclosure is illustrated in FIG. 15. The thermal control unit 22 includes, among other components, reservoir 38, pump 52, heat exchanger 58, manifolds 60, 64, and filter 66.

The following Examples, illustrating the method and system, are intended to illustrate and not limit the present invention.

### EXAMPLES

An ALTRIX™ Precision Temperature Management System (“system”) is provided. The system is available from STRYKER®. The system is generally as set forth in “Operations Manual 8001-009-001 REV D” dated 2015/July, which is incorporated herein by reference in its entirety. The system includes a removable water reservoir. The system has three pairs of output/input ports. The system is not attached to any patient therapy devices.

A disinfectant component is provided. The disinfectant component is commercially available from MEDENTECH® as KLORSEPT™ Bleach Tablets (“tablets”). Each tablet is ˜13.1g in weight and comprises adipic acid, sodium carbonate, and the NaDCC. An aqueous mixture having a volume of 1 gallon is formed by dispersing two tablets in distilled water. After the tablets are fully dissolved, the aqueous mixture has ˜2,000 ppm FAC.

The aqueous mixture is poured into the reservoir of the system. The system is turned on and the aqueous mixture is circulated for ˜15 minutes. Active cooling is used to maintain temperature of the aqueous mixture at ˜25° C. After circulation, the system is deemed to be disinfected. The aqueous mixture is drained from the system. Additional evaluations are described below.

An additional amount of aqueous mixture is formed. Again, the aqueous mixture has ˜2,000 ppm FAC. A gallon of the aqueous mixture is loaded into the system as described above. In certain evaluations, a bypass line is attached to an output port of the system and connected to a corresponding input port of the system to take place of a patient therapy device. This can be done for each pair of ports for disinfection of the same. The aqueous mixture is circulated in the system for ˜15 minutes. In similar evaluations, one end of a bypass line is connected to an outlet port and the other end is dropped into the reservoir. This can be useful for disinfecting the reservoir. In addition, this can be useful for ensuring a near consistent concentration of the disinfectant throughout the system while circulating. In both evaluations, the system is deemed to be disinfected and the aqueous mixture is drained from the system.

In other evaluations, thymol is utilized in place of the tablets to form aqueous mixtures. The thymol is commercially available from Wexford Labs, Inc. as THYMO-CIDE™. An aqueous mixture is formed by dispersing thymol in distilled water. After mixing, the aqueous mixture has ˜130,000 ppm thymol. One gallon loads of the aqueous mixture are loaded and circulated in the system as like above. In each instance, the system is deemed to be disinfected. The aqueous mixture is then drained from the system.

In yet other evaluations, sodium percarbonate is utilized in place of the tablets to form aqueous mixtures. The sodium percarbonate is in the form of granules. An aqueous mixture is formed by dispersing sodium percarbonate in distilled water. The sodium percarbonate is used in such an amount such that after mixing, the aqueous mixture has ˜5,000 ppm H₂O₂. One gallon loads of the aqueous mixture are loaded and circulated in the system as like above. In each instance, the system is deemed to be disinfected. The aqueous mixture is then drained from the system.

In another evaluation, a FLOGENIC® device (“device”; from Medentech Ltd.; see, e.g., the ′557 patent) is connected to an outlet port of the system. Two of the tablets are loaded into the device. The device is connected and situated in such manner as to pour into the reservoir. A gallon of distilled water is poured into the reservoir of the system. The system is then turned on thereby feeding water to the device from the outlet port. The water contacts the tablets in the device to form an aqueous solution. Circulation facilitates dissolution of the tablets until the device is empty of tablets (or residue/particles thereof) and an aqueous solution having ˜2,000 ppm FAC is formed in the system. The aqueous mixture is circulated for ˜10 to 15 minutes. The system is deemed to be disinfected. The aqueous mixture is then drained from the system.

In other evaluations, addition of sodium percarbonate to patient therapy devices is considered. The patient therapy device can be, for example, a wrap, a pad, or a blanket. The patient therapy device is attached to the system and water is circulated within the system and the patient therapy device to refresh the system. In related evaluations, addition of NaDCC to patient therapy devices is considered (in place of sodium percarbonate). It is deemed that effervescence is not required due to the normal circulation of water within the system. The system is refreshed or maintained in these evaluations.

Other disinfectants (and/or disinfectant generators) are evaluated relative to the NaDCC tablets/free-chlorine provided by NaDCC, thymol, and/or sodium percarbonate/H₂O₂. These disinfectants (and/or disinfectant generators) include quaternary ammonium compounds, e.g. MTA33, AIRKEM A-33®, CAVICIDE®, and BURNISHINE®; bleach, e.g. NaOCl; ammonium chloride compounds, e.g. OPTI-CIDE®; chlorine dioxide compounds, e.g. Vital Oxide; and sulfonamide compounds, e.g. Chloramine-T. It was determined that these other disinfectants (and/or disinfectant generators) suffered from one or more deficiencies, including reduced disinfection efficacy, material compatibility issues (e.g. corrosion/break-down of system components), and/or difficulty in handling. In contrast, it was deemed that NaDCC, thymol, and sodium percarbonate/H₂O₂ generally did not suffer from these deficiencies, especially when utilized for disinfection of the system.

The following additional embodiments are provided, the numbering of which is not to be construed as designating levels of importance.

### Additional Embodiments

Embodiment 1 relates to a method of disinfecting a fluid circuit of a thermal control unit for delivering temperature controlled fluid to at least one patient therapy device, said method comprising the steps of: providing an aqueous mixture comprising a disinfectant; and circulating the aqueous mixture in the thermal control unit to disinfect the fluid circuit; wherein the disinfectant comprises free-chlorine, a phenol, H₂O₂, or combinations thereof; and subject to the following provisos; if the disinfectant comprises free-chlorine, the free-chlorine is provided by a chlorinated isocyanurate and is present in the aqueous mixture in an amount of at least about 100 ppm (if the majority or only disinfectant type), if the disinfectant comprises the phenol, the phenol is natural and is present in the aqueous mixture in an amount of at least about 10,000 ppm (if the majority or only disinfectant type), and if the disinfectant comprises H₂O₂, the H₂O₂ is present in the aqueous mixture in an amount of at least about 5,000 ppm (if the majority or only disinfectant type).

Embodiment 2a relates to Embodiment 1, wherein the aqueous mixture comprises about 100 ppm to about 10,000 ppm of free-chlorine.

Embodiment 2b relates to Embodiment 1 or 2a, wherein the aqueous mixture comprises about 2,000 ppm of free-chlorine.

Embodiment 3 relates to any one of the preceding Embodiments, wherein the disinfectant comprises free-chlorine, alternatively consists essentially of free-chlorine, alternatively is free-chlorine.

Embodiment 4 relates to any one of the preceding Embodiments, wherein the free-chlorine comprises HOCl, OCL⁻, or a mixture thereof.

Embodiment 5 relates to any one of the preceding Embodiments, wherein the chlorinated isocyanurate is selected from the group consisting of mono, di and trichloro isocyanurates.

Embodiment 6 relates to Embodiment 5, wherein the chlorinated isocyanurate comprises NaDCC, alternatively consists essentially of NaDCC, alternatively is NaDCC.

Embodiment 7a relates to any one of the preceding Embodiments, wherein the aqueous mixture comprises about 10,000 ppm to about 500,000 ppm of the phenol.

Embodiment 7b relates to any one of the preceding Embodiments, wherein the aqueous mixture comprises about 130,000 ppm of the phenol.

Embodiment 8 relates to any one of the preceding Embodiments, wherein the disinfectant comprises the phenol, alternatively consists essentially of the phenol, alternatively is the phenol.

Embodiment 9 relates to any one of the preceding Embodiments, wherein the phenol comprises thymol.

Embodiment 10a relates to any one of the preceding Embodiments, wherein the aqueous mixture comprises about 5,000 ppm to about 30,000 ppm of the H₂O₂.

Embodiment 10b relates to any one of the preceding Embodiments, wherein the aqueous mixture comprises about 5,000 ppm of the H₂O₂.

Embodiment 11 relates to any one of the preceding Embodiments, wherein the disinfectant comprises H₂O₂, alternatively consists essentially of H₂O₂, alternatively is H₂O₂.

Embodiment 12 relates to any one of the preceding Embodiments, wherein the aqueous mixture has a volume of at least about 1 liter.

Embodiment 13 relates to any one of the preceding Embodiments, wherein the aqueous mixture has a volume of from about 3.75 liters to about 4.25 liters.

Embodiment 14 relates to any one of the preceding Embodiments, wherein the aqueous mixture has a pH of from about 6 to about 9.

Embodiment 15 relates to any one of the preceding Embodiments, wherein the aqueous mixture has a temperature of from about 5° C. to about 95° C.

Embodiment 16 relates to any one of the preceding Embodiments, wherein the aqueous mixture has a temperature of from about 23° C. to about 27° C.

Embodiment 17 relates to any one of the preceding Embodiments, wherein the aqueous mixture consists essentially of the disinfectant and water.

Embodiment 18 relates to any one of Embodiments 1 to 16, wherein the aqueous mixture further comprises an additive selected from the group consisting of surfactants, builders, activators, inhibitors, solubilizers, descalers, chelating agents, acids, bases, water conditioning agents, pH buffers, and combinations thereof.

Embodiment 19 relates to any one of the preceding Embodiments, wherein the aqueous mixture is provided by mixing a disinfectant component with water and wherein the disinfectant component comprises the chlorinated isocyanurate, the phenol, a perhydrate, or combinations thereof.

Embodiment 20 relates to Embodiment 19, wherein the disinfectant component is in the form of a solid, a liquid, or a combination thereof.

Embodiment 21 relates to Embodiment 19 or 20, wherein the disinfectant component comprises NaDCC and/or is in the form of a tablet, granule, powder, or combinations thereof.

Embodiment 22 relates to any one of Embodiments 19 to 21, wherein the disinfectant component further comprises at least one effervescent compound such that the disinfectant component effervesces to facilitate formation of the aqueous mixture.

Embodiment 23 relates to any one of the preceding Embodiments, wherein the aqueous mixture is circulated in the thermal control unit for a least about 5 minutes.

Embodiment 24 relates to any one of the preceding Embodiments, wherein the aqueous mixture is circulated in the thermal control unit for about 10 minutes to about 15 minutes.

Embodiment 25 relates to any one of the preceding Embodiments, further comprising the step of removing the aqueous mixture from the thermal control unit after circulating the aqueous mixture in the thermal control unit to disinfect the fluid circuit.

Embodiment 26 relates to any one of the preceding Embodiments, further comprising the step(s) of: circulating water in the thermal control unit to rinse the fluid circuit of the aqueous mixture or residue thereof; and/or removing the water from the thermal control unit after circulating water in the thermal control unit to rinse the fluid circuit.

Embodiment 27 relates to Embodiment 26, wherein the circulating and removing steps are repeated at least once to further rinse the fluid circuit of the thermal control unit.

Embodiment 28 relates to any one of the preceding Embodiments, wherein a patient is not operatively connected to the thermal control unit, and/or wherein a patient therapy device is not operatively connected to the thermal control unit.

Embodiment 29 relates to any one of Embodiments 25 to 28, wherein the aqueous mixture and the disinfectant is further defined as a first aqueous mixture and a first disinfectant, and further comprising the steps of: providing a second aqueous mixture different from the first aqueous mixture; and circulating the second aqueous mixture in the thermal control unit to substantially maintain disinfection of the fluid circuit; wherein the second aqueous mixture comprises a second disinfectant; wherein the second disinfectant is the same as or different from the first disinfectant; and subject to the following proviso; if the second disinfectant is the same as the first disinfectant, the second disinfectant is present in the second aqueous mixture in an amount less than the amount of the first disinfectant present in the first aqueous mixture.

Embodiment 30 relates to Embodiment 29, wherein the second disinfectant comprises free-chlorine, a phenol, a perhydrate, or combinations thereof.

Embodiment 31 relates to any one of the preceding Embodiments, wherein the aqueous mixture is substantially free of: i) a bleach and free-chlorine provided by a bleach; and/or ii) a sulfonamide and free-chlorine provided by a sulfonamide; and/or iii) a quaternary ammonium compound and free-chlorine provided by a quaternary ammonium compound; and/or iv) a chloramine.

Embodiment 32 relates to any one of the preceding Embodiments, wherein the fluid circuit of the thermal control unit includes an inner surface and wherein the inner surface comprises a material selected from the group consisting of metallic materials, polymeric materials, and combinations thereof.

Embodiment 33 relates to any one of the preceding Embodiments, wherein the fluid circuit of the thermal control unit comprises: a circulation channel for holding a fluid; a pump in fluid communication with the circulation channel for circulating the fluid; an outlet in fluid communication with the circulation channel for sending fluid to at least one patient therapy device; and an inlet in fluid communication with the circulation channel for receiving fluid from the patient therapy device(s); optionally, a bypass line in fluid communication with the outlet and the inlet for allowing circulation of the fluid in the absence of the patient therapy device(s); optionally, at least one supply line in fluid communication with the outlet for sending fluid to the patient therapy devices(s); optionally, at least one supply line in fluid communication with the inlet for receiving fluid from the patient therapy devices(s); and optionally, the patient therapy device(s).

Embodiment 34 relates to Embodiment 33, wherein the thermal control unit further comprises: a heat exchanger operatively connected to the fluid circuit for heating and/or cooling the fluid in the fluid circuit; and a reservoir in fluid communication with the fluid circuit for providing fluid to the fluid circuit; optionally, a separator in fluid communication with the circulation channel for separating entrained air from the fluid; optionally, a filter in fluid communication with the circulation channel for filtering the fluid; and optionally, a controller in electrical communication with at least one of the pump and the heat exchanger for controlling flow and/or temperature of the fluid in the fluid circuit.

Embodiment 35 relates to any one of the preceding Embodiments, wherein the thermal control unit further comprises a dispenser for providing the aqueous mixture comprising the disinfectant.

Embodiment 36 relates to any one of the preceding Embodiments, wherein the thermal control unit further comprises a UV light adjacent the fluid circuit for further disinfecting the fluid circuit, and/or further comprises an ozone generator in fluid communication with the fluid circuit for further disinfecting the fluid circuit.

Embodiment 37 relates to a system comprising: a thermal control unit having a fluid circuit for delivering temperature controlled fluid to at least one patient therapy device; and an aqueous mixture disposed in said fluid circuit; wherein said aqueous mixture comprises a disinfectant for disinfecting said fluid circuit; wherein said disinfectant comprises free-chlorine, a phenol, H₂O₂, or combinations thereof; and subject to the following provisos; if said disinfectant comprises free-chlorine, said free-chlorine is provided by a chlorinated isocyanurate and is present in said aqueous mixture in an amount of at least about 100 ppm, if said disinfectant comprises said phenol, said phenol natural and is present in said aqueous mixture in an amount of at least about 10,000 ppm, and if the disinfectant comprises H₂O₂, the H₂O₂ is present in the aqueous mixture in an amount of at least about 5,000 ppm.

Embodiment 38 relates to Embodiment 37, wherein said fluid circuit of said thermal control unit comprises: a circulation channel for holding a fluid; a pump in fluid communication with said circulation channel for circulating the fluid; an outlet in fluid communication with said circulation channel for sending fluid to at least one patient therapy device; and an inlet in fluid communication with said circulation channel for receiving fluid from the patient therapy device(s); optionally, a bypass line in fluid communication with said outlet and said inlet for allowing circulation of the fluid in the absence of the patient therapy device(s); optionally, at least one supply line in fluid communication with said outlet for sending fluid to the patient therapy devices(s); optionally, at least one supply line in fluid communication with said inlet for receiving fluid from the patient therapy devices(s); and optionally, said patient therapy device(s).

Embodiment 39 relates to Embodiment 38, wherein said thermal control unit further comprises: a heat exchanger operatively connected to said fluid circuit for heating and/or cooling the fluid in said fluid circuit; and a reservoir in fluid communication with said fluid circuit for providing fluid to said fluid circuit; optionally, a separator in fluid communication with said circulation channel for separating entrained air from the fluid; optionally, a filter in fluid communication with said circulation channel for filtering the fluid; and optionally, a controller in electrical communication with at least one of said pump and said heat exchanger for controlling flow and/or temperature of the fluid in said fluid circuit.

Embodiment 40 relates to any one of Embodiments 37 to 39, further comprising a dispenser for providing said aqueous mixture comprising said disinfectant.

Embodiment 41 relates to any one of Embodiments 37 to 40, further comprising a UV light adjacent said fluid circuit for further disinfecting said fluid circuit, and/or further comprising an ozone generator in fluid communication with said fluid circuit for further disinfecting said fluid circuit.

Embodiment 42 relates to any one of Embodiments 37 to 41, wherein the aqueous mixture is as set forth in any one of Embodiments 2 to 31.

Embodiment 43 relates to use of NaDCC to disinfect a fluid circuit of a thermal control unit for delivering temperature controlled fluid to at least one patient therapy device.

Embodiment 44 relates to use of thymol to disinfect a fluid circuit of a thermal control unit for delivering temperature controlled fluid to at least one patient therapy device.

Embodiment 45 relates to use of H₂O₂ to disinfect a fluid circuit of a thermal control unit for delivering temperature controlled fluid to at least one patient therapy device.

Embodiment 46 relates to any one of the preceding Embodiments, wherein at least one of NaDCC, thymol and H₂O₂, alternatively at least one of NaDCC and thymol, alternatively at least NaDCC, is used during a shock (or episodic) portion of the disinfection method.

Embodiment 47 relates to Embodiment 46, wherein at least one of NaDCC, thymol and H₂O₂ is used during a maintenance portion of the disinfection method.

Embodiment 48 relates to any one of the preceding Embodiments, wherein the disinfectant and/or disinfectant component is provided via at least one patient therapy device.

Embodiment 49 relates to any one of the preceding Embodiments, wherein the patient therapy device is selected from the group of wraps, pads, or blankets.

The terms “comprising” or “comprise” are used herein in their broadest sense to mean and encompass the notions of “including,” “include,” “consist(ing) essentially of,” and “consist(ing) of. The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-10, ±0-5, or ±0-2.5, % of the numerical values. Further, The term “about” applies to both numerical values when associated with a range of values. Moreover, the term “about” may apply to numerical values even when not explicitly stated.

Generally, as used herein a hyphen “-” or dash “-” in a range of values is “to” or “through”; a “>” is “above” or “greater-than”; a “≧” is “at least” or “greater-than or equal to”; a “<” is “below” or “less-than”; and a “≦” is “at most” or “less-than or equal to.” On an individual basis, each of the aforementioned applications for patent, patents, and/or patent application publications, is expressly incorporated herein by reference in its entirety in one or more non-limiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

What is claimed is:

1. A method of disinfecting a fluid circuit of a thermal control unit for delivering temperature controlled fluid to at least one patient therapy device, said method comprising the steps of:
providing an aqueous mixture comprising a disinfectant; and
circulating the aqueous mixture in the thermal control unit to disinfect the fluid circuit;
wherein the disinfectant comprises free-chlorine, a phenol, hydrogen peroxide (H₂O₂), or combinations thereof; and
subject to the following provisos;
if the disinfectant comprises free-chlorine, the free-chlorine is provided by a chlorinated isocyanurate and is present in the aqueous mixture in an amount of at least about 100 parts per million (ppm),
if the disinfectant comprises the phenol, the phenol is natural and is present in the aqueous mixture in an amount of at least about 10,000 ppm, and
if the disinfectant comprises H₂O₂, the H₂O₂ is present in the aqueous mixture in an amount of at least about 5,000 ppm.

2. The method as set forth in claim 1, wherein the aqueous mixture comprises about 100 ppm to about 10,000 ppm of free-chlorine, alternatively about 2,000 ppm of free-chlorine.

3. The method as set forth in claim 1, wherein the disinfectant comprises free-chlorine.

4. The method as set forth in claim 3, wherein the free-chlorine comprises hypochlorous acid (HOCl), hypochlorite ions (OCL⁻), or a mixture thereof.

5. The method as set forth in claim 3, wherein the chlorinated isocyanurate is selected from the group consisting of mono, di and trichloro isocyanurates.

6. The method as set forth in claim 5, wherein the chlorinated isocyanurate comprises sodium dichloroisocyanurate (NaDCC).

7. The method as set forth in claim 1, wherein the disinfectant comprises the phenol and wherein the phenol comprises thymol.

8. The method as set forth in claim 7, wherein the aqueous mixture comprises about 10,000 ppm to about 500,000 ppm of the thymol, alternatively about 130,000 ppm of the thymol.

9. The method as set forth in claim 1, wherein the aqueous mixture has a pH of from about 6 to about 9.

10. The method as set forth in claim 1, wherein the aqueous mixture consists essentially of the disinfectant and water.

11. The method as set forth in claim 1, wherein the aqueous mixture further comprises an additive selected from the group consisting of surfactants, builders, activators, inhibitors, solubilizers, descalers, chelating agents, acids, bases, water conditioning agents, pH buffers, and combinations thereof.

12. The method as set forth in claim 1, wherein the aqueous mixture is provided by mixing a disinfectant component with water and wherein the disinfectant component comprises the chlorinated isocyanurate, the phenol, a perhydrate, or combinations thereof.

13. The method as set forth in claim 12, wherein the disinfectant component is in the form of a solid, a liquid, or a combination thereof.

14. The method as set forth in claim 13, wherein the disinfectant component comprises sodium dichloroisocyanurate (NaDCC) and is in the form of a tablet, granule, powder, or combinations thereof.

15. The method as set forth in claim 14, wherein the disinfectant component further comprises at least one effervescent compound such that the disinfectant component effervesces to facilitate formation of the aqueous mixture.

16. The method as set forth in claim 1, wherein:
a patient is not operatively connected to the thermal control unit; and/or
a patient therapy device is not operatively connected to the thermal control unit.

17. The method as set forth in claim 1, wherein the aqueous mixture and the disinfectant is further defined as a first aqueous mixture and a first disinfectant, and further comprising the steps of:
removing the first aqueous mixture from the thermal control unit after circulating the first aqueous mixture in the thermal control unit to disinfect the fluid circuit;
providing a second aqueous mixture different from the first aqueous mixture; and
circulating the second aqueous mixture in the thermal control unit to substantially maintain disinfection of the fluid circuit;
wherein the second aqueous mixture comprises a second disinfectant;
wherein the second disinfectant is the same as or different from the first disinfectant; and subject to the following proviso;
if the second disinfectant is the same as the first disinfectant, the second disinfectant is present in the second aqueous mixture in an amount less than the amount of the first disinfectant present in the first aqueous mixture.

18. The method as set forth in claim 17, wherein the second disinfectant comprises free-chlorine, a phenol, H₂O₂, or combinations thereof.

19. The method as set forth in claim 1, wherein the aqueous mixture is substantially free of:
i) a bleach and free-chlorine provided by a bleach; and/or
ii) a sulfonamide and free-chlorine provided by a sulfonamide; and/or
iii) a quaternary ammonium compound and free-chlorine provided by a quaternary ammonium compound; and/or
iv) a chloramine.

20. The method as set forth in claim 1, wherein the fluid circuit of the thermal control unit includes an inner surface and wherein the inner surface comprises a material selected from the group consisting of metallic materials, polymeric materials, and combinations thereof.

21. The method as set forth in claim 1, wherein the fluid circuit of the thermal control unit comprises:
a circulation channel for holding a fluid;
a pump in fluid communication with the circulation channel for circulating the fluid;
an outlet in fluid communication with the circulation channel for sending fluid to at least one patient therapy device; and
an inlet in fluid communication with the circulation channel for receiving fluid from the patient therapy device(s);
optionally, a bypass line in fluid communication with the outlet and the inlet for allowing circulation of the fluid in the absence of the patient therapy device(s);
optionally, at least one supply line in fluid communication with the outlet for sending fluid to the patient therapy devices(s);
optionally, at least one supply line in fluid communication with the inlet for receiving fluid from the patient therapy devices(s); and
optionally, the patient therapy device(s).

22. The method as set forth in claim 21, wherein the thermal control unit further comprises:
a heat exchanger operatively connected to the fluid circuit for heating and/or cooling the fluid in the fluid circuit; and
a reservoir in fluid communication with the fluid circuit for providing fluid to the fluid circuit;
optionally, a separator in fluid communication with the circulation channel for separating entrained air from the fluid;
optionally, a filter in fluid communication with the circulation channel for filtering the fluid; and
optionally, a controller in electrical communication with at least one of the pump and the heat exchanger for controlling flow and/or temperature of the fluid in the fluid circuit.

23. A system comprising:
a thermal control unit having a fluid circuit for delivering temperature controlled fluid to at least one patient therapy device; and
an aqueous mixture disposed in said fluid circuit;
wherein said aqueous mixture comprises a disinfectant for disinfecting said fluid circuit;
wherein said disinfectant comprises free-chlorine, a phenol, hydrogen peroxide (H₂O₂), or combinations thereof; and
subject to the following provisos;
if said disinfectant comprises free-chlorine, said free-chlorine is provided by a chlorinated isocyanurate and is present in said aqueous mixture in an amount of at least about 100 parts per million (ppm),
if said disinfectant comprises said phenol, said phenol is natural and is present in said aqueous mixture in an amount of at least about 10,000 ppm, and
if the disinfectant comprises H₂O₂, the H₂O₂ is present in the aqueous mixture in an amount of at least about 5,000 ppm.

24. The system as set forth in claim 23, wherein said fluid circuit of said thermal control unit comprises:
a circulation channel for holding a fluid;
a pump in fluid communication with said circulation channel for circulating the fluid;
an outlet in fluid communication with said circulation channel for sending fluid to at least one patient therapy device; and
an inlet in fluid communication with said circulation channel for receiving fluid from the patient therapy device(s);
optionally, a bypass line in fluid communication with said outlet and said inlet for allowing circulation of the fluid in the absence of the patient therapy device(s);
optionally, at least one supply line in fluid communication with said outlet for sending fluid to the patient therapy devices(s);
optionally, at least one supply line in fluid communication with said inlet for receiving fluid from the patient therapy devices(s); and
optionally, said patient therapy device(s).

25. The system as set forth in claim 24, wherein said thermal control unit further comprises:
a heat exchanger operatively connected to said fluid circuit for heating and/or cooling the fluid in said fluid circuit; and
a reservoir in fluid communication with said fluid circuit for providing fluid to said fluid circuit;
optionally, a separator in fluid communication with said circulation channel for separating entrained air from the fluid;
optionally, a filter in fluid communication with said circulation channel for filtering the fluid; and
optionally, a controller in electrical communication with at least one of said pump and said heat exchanger for controlling flow and/or temperature of the fluid in said fluid circuit.
